(19) United States
(12) Patent Application Publication
(10) Pub. No.: US 2026/0297568 A1
(43) Pub. Date: Oct. 1, 2026

(54) METHODS FOR LIGATION-FREE CHROMATIN CONFORMATION CAPTURE WITH HIGH THROUGHPUT SEQUENCING

(71) Applicant: THE TRANSLATIONAL GENOMICS RESEARCH INSTITUTE, Phoenix, AZ (US)

(72) Inventors: Floris P. Barthel, Phoenix, AZ (US); Yi-An Chen, Phoenix, AZ (US); Noelle Fukushima, Phoenix, AZ (US); Ogechukwu Mbegbu, Phoenix, AZ (US)

(21) Appl. No.: 19/479,511

(22) PCT Filed: Apr. 30, 2024
(86) PCT No.: PCT/US2024/027099

§ 371 Date: Oct. 28, 2025

Related U.S. Application Data
(60) Provisional application No. 63/499,237, filed on Apr. 30, 2023.

(51) Int. Cl. C12N 15/10

(52) U.S. Cl. CPC C12N 15/1093

(57) ABSTRACT

Methods are provided to capture telomere-associated DNA loci from chromatin. Chromatin was prepared from cultured cells by dual crosslinking using formaldehyde and EGS, followed by sonication. Biotinylated PNA probes were hybridized to the telomere DNA sequences, and serine protease was used to release the telomere-associated DNA sequences. The released DNA fragments in the supernatant were purified and subjected to library preparation for next-generation sequencing. The ligation-free chromatin capture and sequencing method enriches specific telomere-associated DNA loci to provide an accurate representation of chromatin interaction. Insights into the chromatin interactions occurring at telomeres are used to investigate the potential role of telomers in aging and age-related diseases.

### CROSS-REFERENCE TO RELATED APPLICATIONS

This application claims the benefit of U.S. Provisional App. No. 63/499,237, filed on Apr. 30, 2023, the contents of which are incorporated herein by reference.

### STATEMENT REGARDING FEDERALLY SPONSORED RESEARCH

This invention was made with government support under 5R00CA226387 awarded by the National Institutes of Health. The government has certain rights in this invention.

### INCORPORATION-BY-REFERENCE OF MATERIAL ELECTRONICALLY FILED

The official copy of the sequence listing is submitted electronically in ST.26 XML format having the file name “91482.266WO-PCT_SeqList.xml” created on Apr. 30, 2024, and having a size of 7,084 bytes, and is filed concurrently with the specification. The Sequence Listing ST.26 XML file is part of the specification and is herein incorporated by reference in its entirety.

### FIELD

The present invention relates to the field of methods of identifying chromatin interactions in DNA, and more specifically, to ligation-free chromatin capture methods.

### BACKGROUND

Telomeres are repetitive DNA sequences at the ends of chromosomes that protect the integrity of the genome and shorten with increasing age. Telomeres can silence gene expression by spreading its heterochromatin structure to neighboring regions, i.e., by telomere position effect (TPE) or via long-range chromatin interactions over larger distances, i.e., TPE over long distances (TPE-OLD). Recent studies have nominated genes in human cells controlled by TPE-OLD in a telomere length-dependent manner, suggesting that TPE-OLD is an epigenetic mechanism of transcriptional regulation important in aging and age-related disease. However, the larger-scale structure of telomeres and their interactions with the rest of the genome remain largely unknown.

A current standard for validation of telomeric chromatin interaction is telomere 3D in situ hybridization. However, its scale is limited by the number of probes applied in each experiment.

### SUMMARY

A need exists to determine the underlying molecular mechanisms regulating telomerase reverse transcriptase (TERT) expression in cancer and other diseases. To fulfill the emerging demand for large-scale studies of telomeric chromatin interactions, the present disclosure includes methods of telomere chromatin conformation capture with high throughput sequencing for this purpose.

Telomeric chromatin is densely packed with nucleosomes and shelterin. Telomeres can influence gene expression by forming long-range chromatin loops (telomere position effect over long distances, or TPE-OLD). Telomerase reactivation is a fundamental event in the genesis of nearly every human cancer. Although transcriptionally silent in differentiated adult cells, its catalytic component telomerase reverse transcriptase (TERT) is expressed in over 80% of human cancers. Despite the recent discovery of reactivating TERT promoter mutations, very little is known about mechanisms leading to the reactivation of telomerase in human cancer.

This disclosure provides methods for isolating associated DNA from reverse crosslinked supernatant to achieve a ligation-free library preparation. The disclosed methods further capture telomere-associated complexes from crosslinked chromatin and sequence them using high-throughput sequencing.

These methods enable identification of locus-to-locus interaction in a 3D genomic manner. Unlike other 3D genomic technologies, the present method is ligation-free and utilizes probes to capture chromatin with specific loci DNA. This method enables the enrichment of any desired locus and its interacting loci, offering researchers a robust tool for studying the interactions between two loci, providing a more accurate representation of chromatin interaction. The ability to enrich specific loci with this technology has the potential to offer new insights into gene regulation, chromatin structure, and genome function, making it a promising technique for researchers in a variety of fields.

In various embodiments, the method of enriching a DNA locus in chromatin may include the steps of preparing chromatin by dual crosslinking using methanol-free formaldehyde and ethylene glycol bis(succinimidyl succinate) (EGS) to produce dual crosslinked chromatin, and shearing the dual crosslinked chromatin using sonication. The method may include capturing PNA probes and a set of DNA sequences from the chromatin by, (a) hybridizing peptide nucleic acid (PNA) probes with attached biotinylated beads to DNA sequences in the dual crosslinked chromatin, (b) reversing the dual crosslink in the chromatin using a protein digestion enzyme to release the DNA sequences to produce released DNA sequences, and (c) separating the released DNA sequences by applying a magnet to the biotinylated beads. The method may further include the step of extracting from the mixture a fluid (supernatant) containing the released DNA sequences. The fluid does not contain the PNA probes or biotinylated beads. The method may further include the steps of purifying the released DNA sequences, and generating a ligation-free library with the released DNA sequences. The released DNA sequences include the DNA locus. The method may further include the steps of indexing the ligation-free library, and sequencing the ligation-free library using next generation sequencing.

In some aspects, the disclosure provides a method of enriching a DNA locus in chromatin, comprising: preparing chromatin by dual crosslinking using a nucleic acid crosslinking agent and a protein crosslinking agent to produce dual crosslinked chromatin; shearing the dual crosslinked chromatin using enzymatic digestion or mechanical force; capturing peptide nucleic acid (PNA) probes and a set of DNA sequences from the chromatin by: (a) hybridizing PNA probes with attached biotinylated beads to DNA sequences in the dual crosslinked chromatin, (b) reversing the dual crosslink in the chromatin using a protein digestion enzyme to release the DNA sequences to produce released DNA sequences, and (c) separating the released DNA sequences by applying a magnet to the biotinylated beads; extracting from the mixture a fluid containing the released DNA sequences, wherein the fluid does not contain the PNA probes or biotinylated beads; purifying the released DNA sequences; generating a ligation-free library with the released DNA sequences, wherein the released DNA sequences include the DNA locus; indexing the ligation-free library; and sequencing the ligation-free library using next generation sequencing.

In some aspects, the protein crosslinking agent is selected from the group consisting of bis(sulfosuccinimidyl) suberate (BS3), disuccinimidyl suberate (DSS), dimethylsuberimidate (DMS), formalin, dimethyladipimidate (DMA), dithio-bis(-succinimidyl propionate) (DSP), disuccinimidyl tartrate (DST), N-hydroxysuccinimide, N-hydroxysulfosuccinimide, ethylene glycol bis(succinimidyl succinate) (EGS), sulfo-ethylene glycol bis(succinimidylsuccinate) (sulfo-EGS), ethylene glycol bis(sulfosuccinimidyl succinate) (SEGS), glutaraldehyde, polyfunctional aziridine, bifunctional carbodiimide, dicyclohexyl carbodiimide, and bis-imidazole-carboxylate compounds.

In other aspects, the nucleic crosslinking agent is selected from the group consisting of glutaraldehyde, formaldehyde, psoralen, aminomethyltrioxsalen, cisplatin, disuccinimidyl glutarate, formalin, UV light, mitomycin C, nitrogen mustard, melphalan, 1,3-butadiene diepoxide, cis diaminedichloroplatinum(II), and cyclophosphamide.

In one aspect, the protein crosslinking agent is EGS. In another aspect, the nucleic crosslinking agent is methanol-free formaldehyde. In some aspects, the dual crosslinked chromatin is sheared with mechanical force using sonication, nebulization, hydrodynamic shearing, syringe pumping.

In other aspects, the protein digestion enzyme is a serine protease, cysteine protease, aspartic protease, threonine protease, metalloprotease, glutamic protease, thiol protease, or a combination thereof. In one aspect, the protein digestion enzyme is a serine protease.

In some aspects, the released DNA sequences are associated with a transposable element or interspersed repeat. In one aspect, the transposable element or interspersed repeat is selected from the list presented in FIG. 14. In another aspect, the transposable element or interspersed repeat is a telomere-associated DNA sequence. In some aspects, the telomere-associated DNA sequence is a Telomere-Associated Repeat Element (TARE), Subtelomeric Repeat, Interstitial Telomeric Sequence (ITS), Short Interspersed Nuclear Element (SINE), Long Interspersed Nuclear Element (LINE), or satellite DNA. In another aspect, the telomere-associated DNA sequence is (CCCTAA)n or (TTAGGG)n.

In other aspects, the released DNA sequences are associated with a Retrotransposable Element (RE). In one aspect, the RE is an Alu element (ALU). In some aspects, the ALU belongs to a subfamily selected from the group consisting of AluJ, AluS, AluY, AluSc, AluSq, AluSp, AluYb8, AluYa5, AluYh9, and AluJo.

In other aspects, the PNA probe comprises a complementary nucleotide sequence to a transposable element, interspersed repeat, or retrotransposable element.

In some aspects, the disclosure provides a method of capturing telomere-associated DNA loci from chromatin, comprising: crosslinking the chromatin by mixing chromatin with a nucleic acid crosslinking agent and a protein crosslinking agent to produce dual crosslinked chromatin; shearing the dual crosslinked chromatin using enzymatic digestion or mechanical force; capturing PNA probes and a set of DNA sequences from the chromatin by: (a) hybridizing PNA probes with biotinylated beads to DNA sequences in the dual crosslinked chromatin, (b) reversing the dual crosslink in the chromatin using a protein digestion enzyme to release the DNA sequences to produce released DNA sequences, and (c) separating the released DNA sequences by applying a magnet to the biotinylated beads; and extracting from the mixture a fluid containing the released DNA sequences, wherein the fluid does not contain the PNA probes or biotinylated beads, and wherein the released DNA sequences include the telomere-associated DNA loci.

This disclosure outlines a novel methodology utilizing a single probe targeting a transposable element, interspersed repeat, or retrotransposable element, resulting in a more cost-efficient approach compared to existing techniques. Additionally, the disclosed method involves the elimination of the repeated element captured by the probe upon assay completion, consequently simplifying the resulting associated DNA fragments. This reduction in complexity significantly streamlines the subsequent computational analysis.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the figures, wherein like numerals may denote like elements.

FIG. 1 illustrates principals and methods of ligation-free chromatin conformation capture with high throughput sequencing;

FIG. 2 illustrates details of the PNA capture probe step from FIG. 1;

FIG. 3 illustrates principals and methods of ligation-free chromatin conformation capture with high throughput sequencing;

FIGS. 4A-4B illustrate results showing negative association between the method's signals and chromosome ends;

FIGS. 5A-5C illustrate results showing telomeric chromatin interacts in the loci of TERT, CCND2, ITS, and at consensus sites;

FIGS. 6A-6D illustrate, at the end of chromosomes, the periodicity of the signal produced by the disclosed ligation-free chromatin capture and sequencing method;

FIGS. 7A-7C illustrate enrichment of the signal produced by the disclosed ligation-free chromatin capture and sequencing method in ITS regions;

FIG. 8 illustrates a representation of a telomer; and

FIGS. 9A-9C illustrate example fragment yields and size quality used in the sonication quality control (QC) step of the ligation-free chromatin capture and sequencing method.

FIG. 10 illustrates detection of proximal and distal telomere-chromatin interaction in Chr20 by Telomere-C.

FIGS. 11A-11C illustrate Telomere-C signals are rich in the Telomere Associated Repeat 1, (CCCTAA)n, or (TTAGGG)n, and other repeat elements.

FIGS. 12A and 12B illustrate validation of Telomere-ITSs interaction by 3C.

FIGS. 13A and 13B illustrate detection of a trans Telomere-ITS interaction by fluorescence in situ hybridization (FISH).

FIG. 14 illustrates a list of transposable elements or interspersed repeats that can be analyzed with the disclosed methods.

### DETAILED DESCRIPTION

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

As used herein, “amplification reaction” refers to a method of detecting target nucleic acid by in vitro amplification of DNA or RNA.

As used herein, “polymerase chain reaction (PCR)” refers to the amplification of a specific DNA sequence, termed target or template sequence, that is present in a mixture, by adding two or more short oligonucleotides, also called primers, that are specific for the terminal or outer limits of the template sequence. The template-primers mixture is subjected to repeated cycles of heating to separate (melt) the double-stranded DNA and cooling in the presence of nucleotides and DNA polymerase such that the template sequence is copied at each cycle.

The term “primer” refers to DNA oligonucleotides complementary to a region of DNA and serves as the initiation of amplification reaction from the 5′ to 3′ direction. For example, a forward and a reverse marker-specific primer can be designed to amplify the marker from a nucleic acid sample.

The term “primer pair” refers to the forward and reverse primers in an amplification reaction leading to amplification of a double-stranded DNA region of the target.

The term “target” refers to a nucleic acid region bound by a primer pair that is amplified through an amplification reaction. The PCR “product” or “amplicon” is the amplified nucleic acid resulting from PCR of a set of primer pairs.

The term “multiplex amplification reaction” herein refers to the detection of more than one template in a mixture by the addition of more than one set of oligonucleotide primers.

“Amplification” is a special case of nucleic acid replication involving template specificity. Amplification may be a template-specific replication or a non-template-specific replication (i.e., replication may be specific template-dependent or not). Template specificity is here distinguished from fidelity of replication (synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out. The amplification process may result in the production of one or more amplicons.

The term “amplifiable nucleic acid” refers to nucleic acids that may be amplified by any amplification method. It is contemplated that “amplifiable nucleic acid” will usually comprise “sample template.” The terms “PCR product,” “PCR fragment,” “amplification product,” and “amplicon” refer to the resultant mixture of compounds after two or more cycles of the PCR steps of denaturation, annealing and extension. These terms encompass the case where there has been amplification of one or more segments of one or more target sequences.

PCR generally involves the mixing of a nucleic acid sample, two or more primers or oligonucleotides (primers and oligonucleotides are used interchangeably herein) that are designed to recognize the template DNA, a DNA polymerase, which may be a thermostable DNA polymerase such as Taq or Pfu, and deoxyribose nucleoside triphosphates (dNTP's). In some embodiments, the DNA polymerase used can comprise a high fidelity Taq polymerase such that the error rate of incorrect incorporation of dNTPs is less than one per 1,000 base pairs. Reverse transcription PCR, quantitative reverse transcription PCR, and quantitative real time reverse transcription PCR are other specific examples of PCR. In general, the reaction mixture is subjected to temperature cycles comprising a denaturation stage (typically 80-100° C.), an annealing stage with a temperature that is selected based on the melting temperature (Tm) of the primers and the degeneracy of the primers, and an extension stage (for example 40-75° C.).

Detection according to some embodiments of the disclosure may comprise contacting the amplified nucleic acid with a probe; and detecting the hybridization of probe with the amplified nucleic acid. Detection may be performed by a variety of methods, such as but not limited to, by a nucleic acid amplification reaction. In some embodiments the amplification reaction maybe an end-point determination or the amplification reaction maybe quantitative. The quantification may be a real-time PCR method. In some embodiments, the real-time PCR may be a SYBRR Green Assay or a TAQMAN® Assay. Detection, in various embodiments, maybe performed by hybridization using probes specific to target sequences. According to various embodiments, combinations of amplification and hybridization may be used for detection.

A marker may be any molecular structure produced by a cell, expressed inside the cell, accessible on the cell surface, or secreted by the cell. A marker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination. A marker may also be called a target and the terms are used interchangeably.

A marker may be represented by the sequence of a nucleic acid from which it can be derived or any other chemical structure. Examples of such nucleic acids include miRNA, tRNA, siRNA, mRNA, cDNA, or genomic DNA sequences including complimentary sequences. Alternatively, a marker may be represented by a protein sequence. The concept of a marker is not limited to the products of the exact nucleic acid sequence or protein sequence by which it may be represented. Rather, a marker encompasses all molecules that may be detected by a method of assessing the expression of the marker.

Expression encompasses any and all processes through which material derived from a nucleic acid template may be produced. Expression thus includes processes such as RNA transcription, mRNA splicing, protein translation, protein folding, post-translational modification, membrane transport, associations with other molecules, addition of carbohydrate moieties to proteins, phosphorylation, protein complex formation and any other process along a continuum that results in biological material derived from genetic material whether in vitro, in vivo, or ex vivo. Expression also encompasses all processes through which the production of material derived from a nucleic acid template may be actively or passively suppressed. Such processes include all aspects of transcriptional and translational regulation. Examples include heterochromatic silencing, transcription factor inhibition, any form of RNAi silencing, microRNA silencing, alternative splicing, protease digestion, posttranslational modification, and alternative protein folding.

Expression may be assessed by any number of methods used to detect material derived from a nucleic acid template used currently in the art and yet to be developed. Examples of such methods include any nucleic acid detection method including the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing, direct sequencing of genomic DNA, or any other method of detecting a specific nucleic acid now known or yet to be disclosed. Other examples include any process of assessing protein expression including flow cytometry, immunohistochemistry, ELISA, Western blot, and immunoaffinity chromatography, HPLC, mass spectrometry, protein microarray analysis, PAGE analysis, isoelectric focusing, 2-D gel electrophoresis, or any enzymatic assay.

The term “library,” as used herein refers to a library of genome/transcriptome-derived sequences. The library may also have sequences allowing amplification of the “library” by the polymerase chain reaction or other in vitro amplification methods well known to those skilled in the art. In various embodiments, the library may have sequences that are compatible with next-generation high throughput sequencing platforms.

In eukaryotes, genomic DNA is packed into chromatin as chromosomes within the nucleus. The basic structural unit of eukaryotic native chromatin is the nucleosome, which consists of 146 base pairs (bp) of DNA wrapped around a histone octamer. The histone octamer consists of two copies each of the core histone H2A-H2B dimers and H3-H4 dimers. Nucleosomes are regularly spaced along the DNA in what is commonly referred to as “beads on a string”.

The assembly of core histones and DNA into nucleosomes is mediated by chaperone proteins and associated assembly factors. Nearly all of these factors are core histone-binding proteins. Some of the histone chaperones, such as nucleosome assembly protein-1 (NAP-1), exhibit a preference for binding to histones H3 and H4. It has also been observed that newly synthesized histones are acetylated and then subsequently deacetylated after assembly into chromatin. The factors that mediate histone acetylation or deacetylation therefore play an important role in the chromatin assembly process.

In general, two in vitro methods have been developed for reconstituting or assembling chromatin. One method is ATP-independent, while the second is ATP-dependent. The ATP-independent method for reconstituting chromatin involves the DNA and core histones plus either a protein like NAP-1 or salt to act as a histone chaperone. This method results in a random arrangement of histones on the DNA that does not accurately mimic the native core nucleosome particle in the cell. These particles are often referred to as mononucleosomes because they are not regularly ordered, extended nucleosome arrays and the DNA sequence used is usually not longer than 250 bp (Kundu, T. K. et al., Mol. Cell 6:551-561, 2000). To generate an extended array of ordered nucleosomes on a greater length of DNA sequence, the chromatin must be assembled through an ATP-dependent process.

The ATP-dependent assembly of periodic nucleosome arrays, which are similar to those seen in native chromatin, requires the DNA sequence, core histone particles, a chaperone protein and ATP-utilizing chromatin assembly factors. ACF (ATP-utilizing chromatin assembly and remodeling factor) or RSF (remodeling and spacing factor) are two widely researched assembly factors that are used to generate extended ordered arrays of nucleosomes into chromatin in vitro (Fyodorov, D. V., and Kadonaga, J. T. Method Enzymol. 371:499-515, 2003; Kundu, T. K. et al. Mol. Cell 6:551-561, 2000).

In particular embodiments, the methods of the disclosure can be easily applied to any type of fragmented double stranded DNA crosslinked to chromatin. Examples include but are not limited to free DNA isolated from plasma, serum, and/or urine; and apoptotic DNA from cells and/or tissues. The DNA can be fragmented enzymatically in vitro (for example, by DNase I, transposase, and/or restriction endonuclease) or fragmented by mechanical forces (hydro-shear, sonication, nebulization, etc.).

Fragmentation of the double stranded DNA can be accomplished by any of the following methods which provide a certain degree over the fragment size:
Enzymatic Digestion (Restriction Enzymes): With this method, restriction enzymes, also known as restriction endonucleases, recognize and cut DNA at specific sequences. The cut sites are predictable, allowing for control over the fragment sizes to some extent.
Micrococcal Nuclease Digestion: Micrococcal nuclease preferentially cuts the DNA at regions that are not protected by proteins, such as nucleosomes. The digestion can be controlled by time and enzyme concentration to achieve the desired fragment size.
DNase I Digestion: DNase I cuts DNA non-specifically. By adjusting the enzyme concentration and the incubation time, it is possible to partially digest the DNA to obtain fragments of a certain size range.
Chemical Cleavage: Chemical agents like hydroxyl radicals, copper ions, or other chemical nucleases can be used to cleave DNA. The size of the fragments can be regulated by the concentration of the chemicals and the duration of the exposure.
Mechanical Shearing: Apart from sonication, DNA can be mechanically sheared through passing it through narrow orifices or needles at high pressure, using devices specifically designed for this purpose.
Nebulization: DNA is forced through a small hole in a nebulizer device, and the shear forces as the DNA exits the hole create the fragments. The size can be controlled by the pressure and the time of nebulization.
Hydrodynamic Shearing: DNA is sheared by forces exerted in a fluid flow, such as those created in specific devices like hydro-shear or g-TUBE.
Pointed-end Needles or Syringe Pumping: Passing DNA solution through narrow-gauge needles or using a syringe pump can create shear forces sufficient to break the DNA into smaller fragments.
Laser Fragmentation: Short laser pulses can be used to break down DNA into fragments. The energy and frequency of the laser can be controlled to achieve the desired fragment size.
Automated Fragmentation Devices: There are automated systems like the COVARIS® system that use adaptive focused acoustics to shear DNA into fragments of controlled size.

Methylation of a region in the TERT promoter is shown to be associated with increased transcription. The present method was developed to provide methods of epigenetic editing to measure the impact of manipulating DNA methylation on telomerase activity. The present approach included confirming that DNA methylation is responsible for reactivating telomerase in cancer development. One goal of this approach was to assess molecular mechanisms that are affected by methylation at the TERT promoter. Another goal was to determine whether chromatin conformation and transcription factor (TF) binding are regulated via methylation. Yet another goal was to identify what changes in DNA methylation, TF binding and chromatin conformation are associated with telomerase reactivation in cancer development. These stated goals are aimed at establishing the role of methylation in reactivating telomerase in cancer and providing insights into the nature of its spontaneous reactivation, and ultimately, to identify vulnerabilities of telomerase reactivation during cancer development.

In some aspects, the present approach captures telomere-associated complexes from crosslinked chromatin and sequences them using high-throughput sequencing.

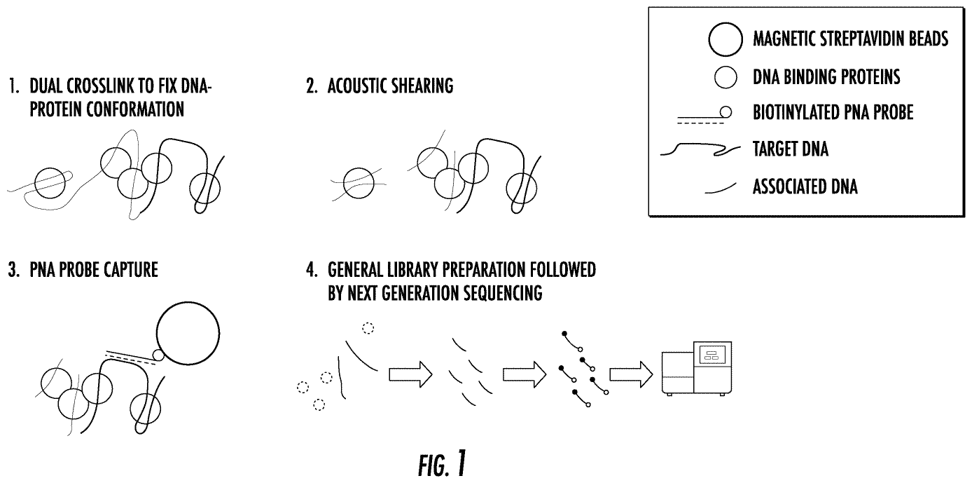

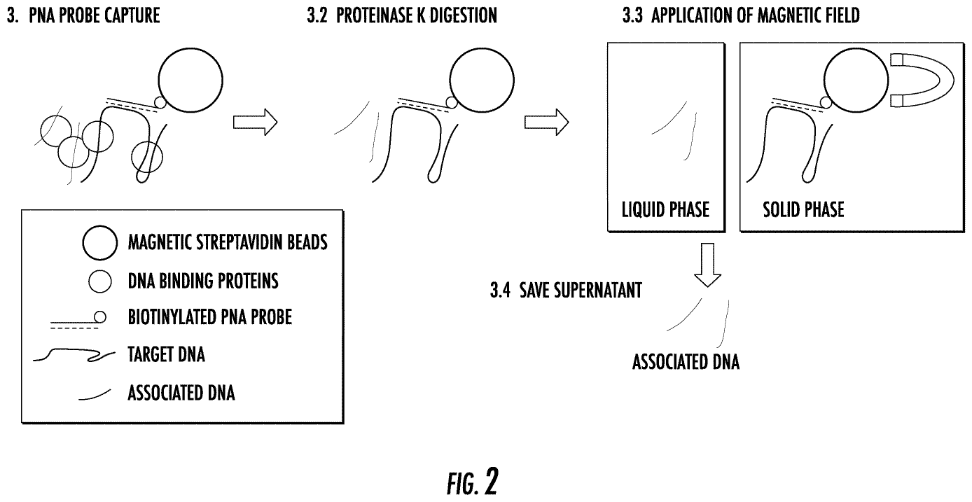

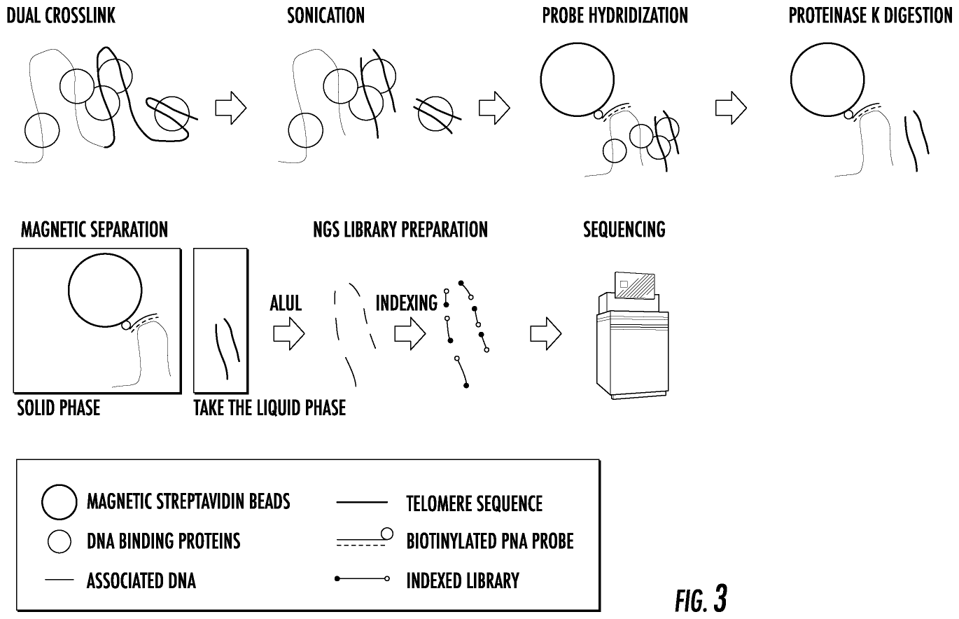

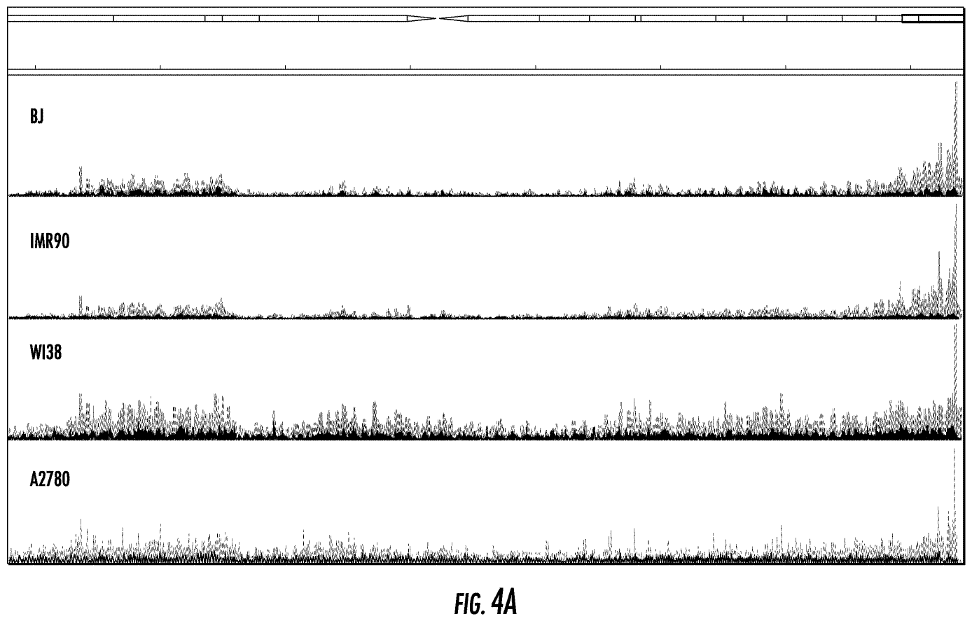

FIGS. 1-3 show principles of the present method. Chromatin was prepared from cultured cells by dual crosslinking using formaldehyde and EGS, followed by sonication. Biotinylated PNA probes were hybridized to the telomere sequences, and proteinase K was used to release the telomere-associated sequences. The released DNA sequences were separated from the mixture using streptavidin magnetic beads. The isolated DNA sequences were used to generate the NGS library for high-throughput sequencing.

Additional nucleic acid crosslinkers that can be used with the disclosed methods are known to one of skill in the art. See, e.g., Harris and Christian, Meth. Enzymol. 468:127-146, 2009. In some examples, a cross linker is light activated. These crosslinkers include formaldehyde, disuccinimidyl glutarate, UV-254, psoralens and their derivatives such as aminomethyltrioxsalen, glutaraldehyde, ethylene glycol bis[succinimidylsuccinate], bissulfosuccinimidyl suberate, 1-Ethyl-3-[3-dimethylaminopropyl]carbodiimide (EDC) bis[sulfosuccinimidyl] suberate (BS3) and other compounds known to those skilled in the art, including those described in the Thermo Scientific Pierce Crosslinking Technical Handbook.

The method involves the use of a peptide nucleic acid (PNA) probe to specifically hybridize DNA sequences involved in chromatin interactions, which are then captured using biotinylated beads. The bead-bound chromatin is then decrosslinked using proteinase K, a serine protease, and the released DNA fragments in the supernatant are purified and subjected to library preparation for next-generation sequencing.

In some embodiments, sheared chromatin is incubated with 0.25 μM PNA probe with the following hybridization program: a. 25° C. for 3 min b. 71° C. for 9 min c. 38° C. for 1 h d. 25° C. final temperature. Dynabeads MyOne T1 are added to the mix and incubated with probe-hybridized chromatin for 2 hours at room temperature followed by wash steps. After the wash steps, the bead-bound chromatin is resuspended in TE buffer. Proteinase K is added and the mix is incubated at 65° C. for 4 hours. The mix is placed on the magnetic stand, and only the supernatant is transferred to a new tube, not the beads. The supernatant is then purified followed by AluI digestion, and regular NGS library preparation is performed.

Results herein show that the signal resulting from the ligation-free chromatin capture and sequencing method is strongest near chromosome ends and gradually declines over the length of a chromosome arm. Moreover, the resulting signal shows a striking periodicity, suggestive of regularly spaced chromatin loops. The signal is markedly pronounced at sites of interstitial telomeric sequences (ITS), potentially representing chromatin hubs of multiple interacting telomeric loci. Interestingly, a Telomere-C peak was found in the vicinity of the TERT locus, which could point to a telomere-length dependent auto-feedback loop controlling TERT transcription. These findings provide insights into chromatin interactions occurring at telomeres and their role in cancer and aging. This approach provides a new toolkit to study the higher-order chromatin structure of telomeres and their relationship to the rest of the genome.

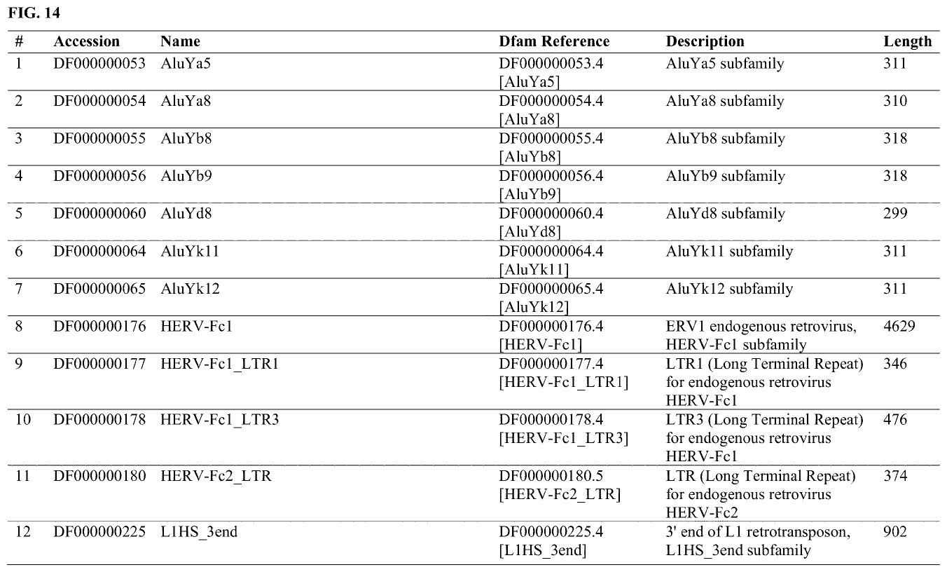

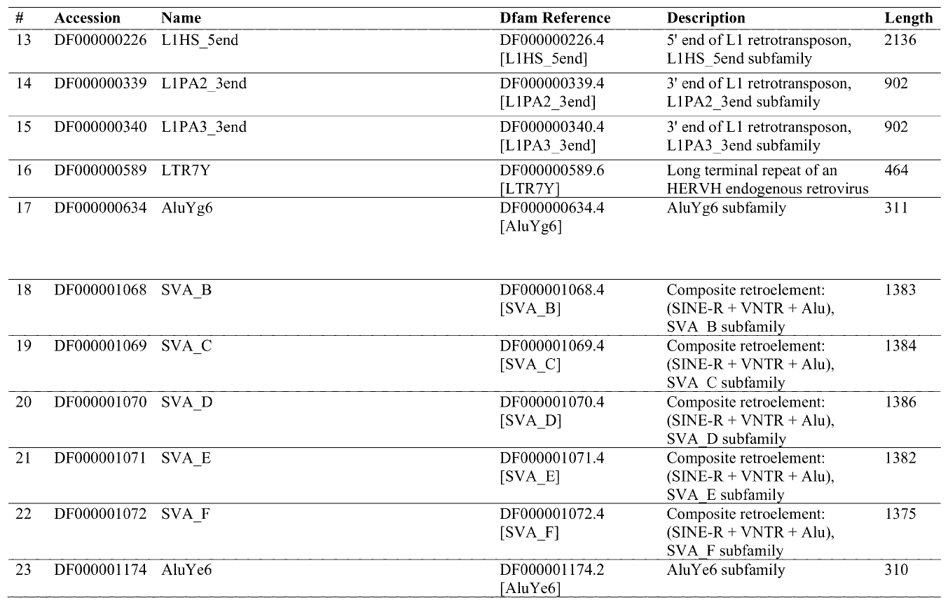

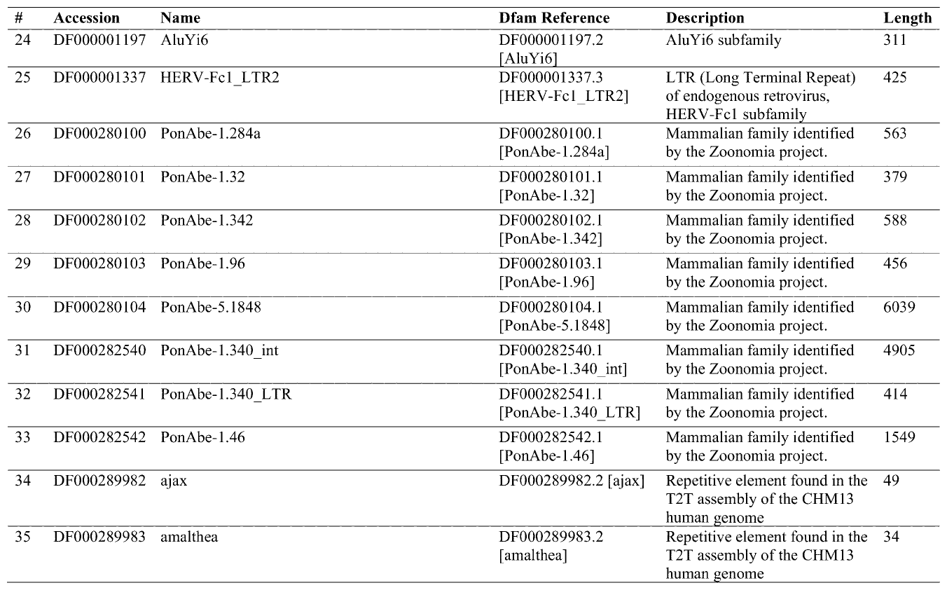

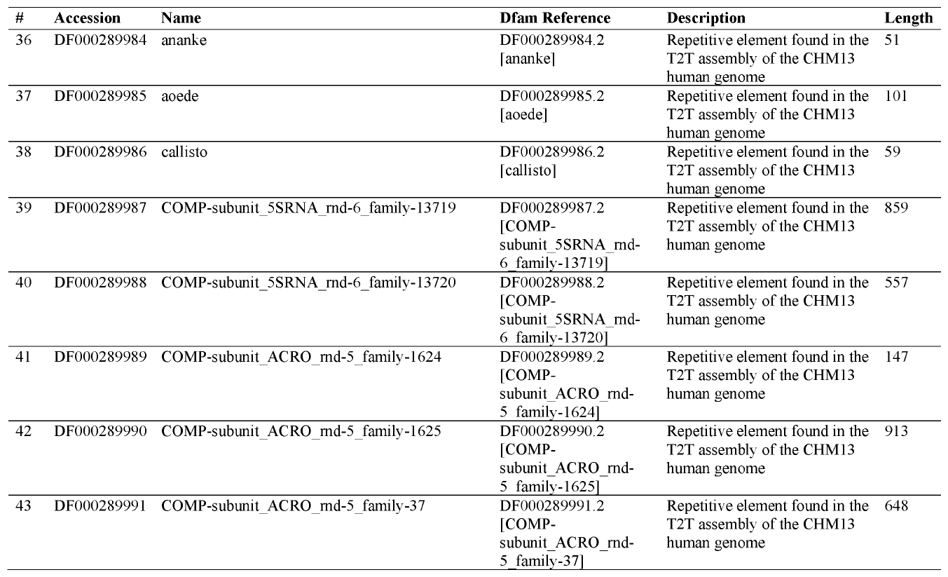

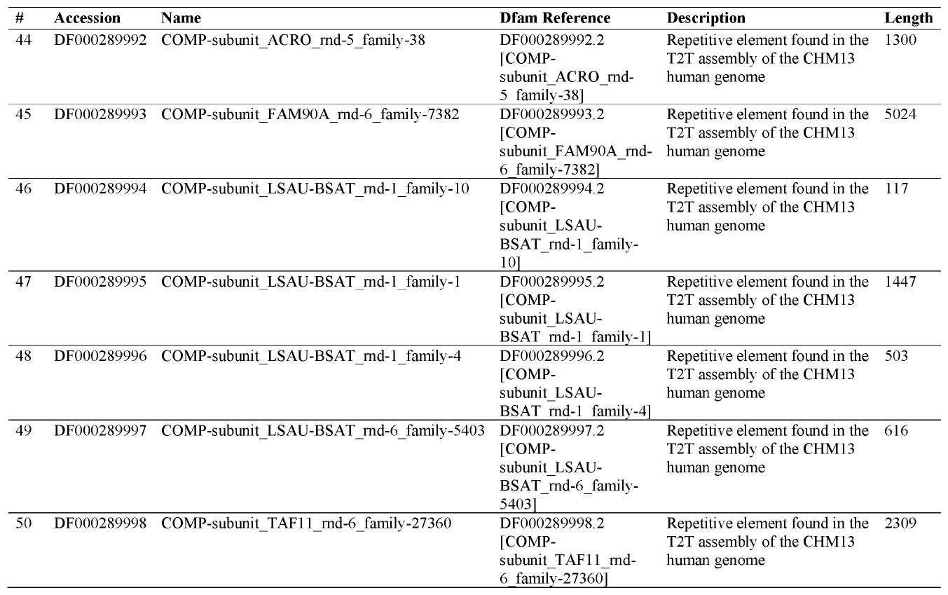

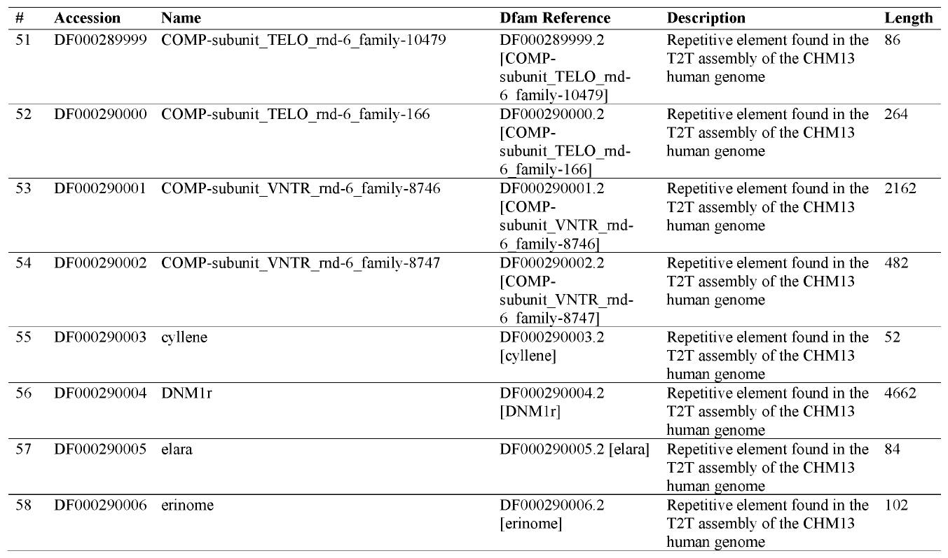

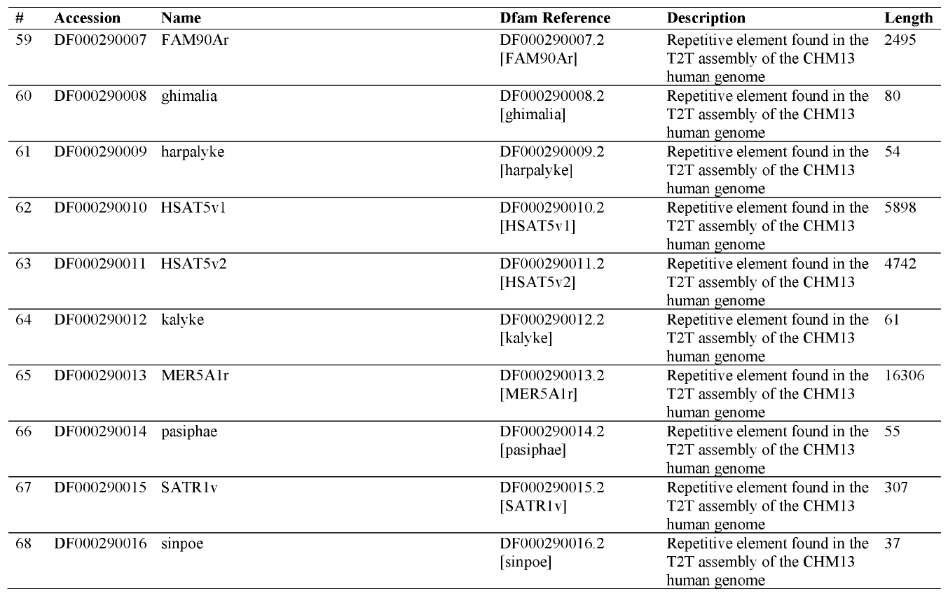

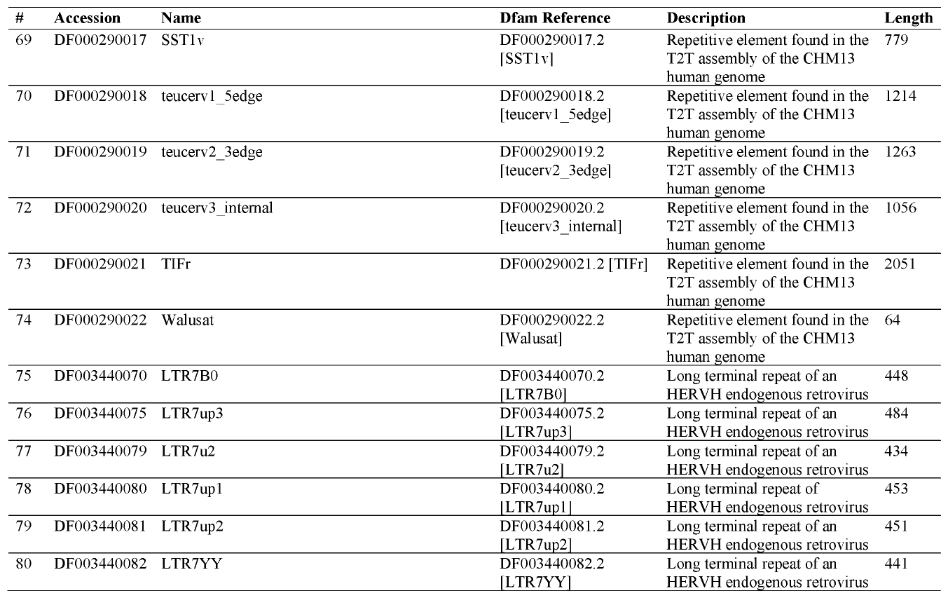

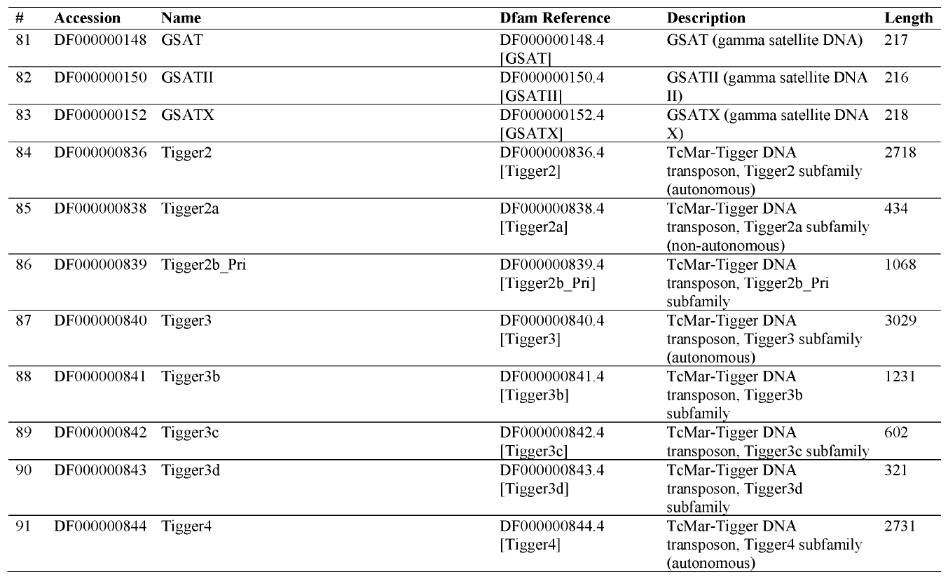

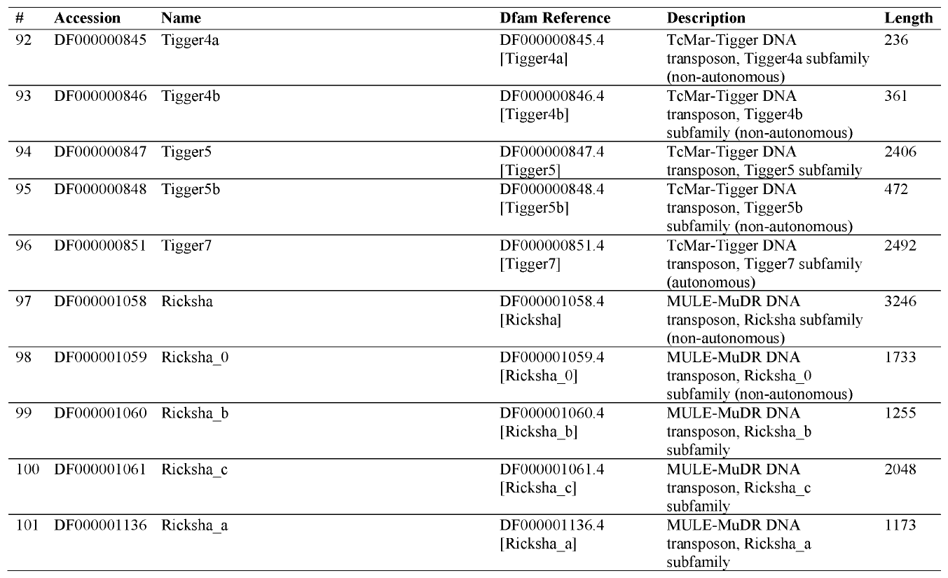

While the disclosed methods can be used to analyze interactions between repeating elements in telomeric sequences and associated segments of genomic DNA, the methods are not limited to this application. In other aspects, the disclosed methods are used to analyze the interactions of transposable elements or interspersed repeats present in a genome with associated segments of genomic DNA. A non-limiting list of such transposable elements or interspersed repeats is available in the Dfam database. In one aspect, the disclosed methods are used to analyze associations between one or more of the transposable elements or interspersed repeats listed in FIG. 14 and associated segments of genomic DNA.

In yet other aspects, the disclosed methods are used to analyze the interactions of Retrotransposable Elements (REs) present in a genome with associated segments of genomic DNA. Retrotransposable Elements (REs) are mobile element insertion polymorphisms that are essentially homoplasy-free characters, identical by descent and easy to genotype (reviewed in Batzer M A; Deininger, P L, Alu repeats and human genomic diversity, Nat. Rev. Genet. 3 (5): 370-9 (2002), doi: 10.1038/nrg798). ALUs are REs that are approximately 300 bp insertions and are distributed throughout the human genome in large copy number. In addition to the major retrotransposon families, REs include smaller families of transposons such as SVA or long interspersed element (“LINE”). SVA elements, named after its main components, short interspersed element (“SINE”), variable number tandem repeat (“VNTR”) and Alu element (“ALU”), contain the hallmarks of retrotransposons, in that they are flanked by target site duplications (“TSDs”), terminate in a poly(A) tail and they are occasionally truncated and inverted during their integration into the genome (Ono, M; Kawakami, M; Takezawa, T, A novel human nonviral retroposon derived from an endogenous retrovirus. Nucleic Acids Res. 15 (21): 8725-8737 (1987); Wang, H, et al., SVA elements: A hominid-specific retroposon family, J. Mol. Biol. 354 (4): 994-1007 (2005), doi: 10.1016/j.jmb.2005.09.085). Long-Interspersed Elements (LINE1) are similar to ALU and SVA in that they also contain the hallmarks of retrotransposons and are high copy number, but differ in size, being up to several kilobases in length (Deininger, P L: Batzer, M A, Mammalian Retroelements. Genome Res. 12 (10): 1455-65 (2002), doi: 10.1101/gr.282402).

The following examples are given for purely illustrative and non-limiting purposes of the present invention.

### EXAMPLES

In these methods, telomere-associated complexes are captured by a biotinylated telomeric PNA probe from ~10 μg of dual crosslinked chromatin, followed by reverse crosslinking and library preparation. These methods aim to obtain expression data to measure associated changes in transcription. The following describes an exemplary protocol for the ligation-free chromatin capture and sequencing method.

### Cell Harvesting and Dual Cross-Linking

Chromatin crosslinking is the fundamental step to fix contact information. In this protocol, EGS works for protein-protein crosslinking; while formaldehyde works for protein-DNA crosslinking. However, over-crosslinking makes chromatin resistant to shearing by sonication. To get optimal results, use methanol-free formaldehyde because methanol reduces membrane permeability causing resistance of shearing by sonication.

1. Freshly prepare EGS fixative solution in a fume hood by dissolving 0.02 g EGS (Ethylene glycol bis[succinimidylsuccinate]) in 200 μl DMSO at 37° C. for 5 minutes (220 mM final concentration). 2. Mix 200 μl EGS/DMSO with 29.8 ml 1×PBS, and then place the EGS mixture at 37° C. until use (1.5 mM final concentration). Unlike Hi-C, the method performs dual crosslink by adding EGS for crosslink protein-protein interaction preserving better chromatin conformation than single crosslink (i.e., single crosslink refers to using formaldehyde without using EGS). 3. Collect cells (~100 million), cultured to ~80% confluency under recommended conditions, by centrifugation at 200×g for 5 minutes at room temperature. Discard the medium and wash the cell pellet with 10 ml 1×PBS buffer once. Centrifuge at 200×g for 5 minutes at room temperature and discard 1×PBS buffer. 4. Resuspend cell pellet in 30 ml freshly prepared EGS fixative solution and mix the cell with rotation (10 rpm) for 30 minutes at room temperature. (Depends on cell lines, incubation time can be from 20 min to 45 min). 5. Add 2 ml 16% (vol/vol) formaldehyde to the fixative solution to a final concentration of 1% (vol/vol) and mix with rotation (10 rpm) for 10 minutes at room temperature. Depending on cell lines, incubation time can be from 5 min to 20 min. 6. Quench cross-linking reaction by adding 2.78 ml 2.5M glycine to the fixative solution to a final concentration of 0.2 M with rotation (10 rpm) for 5 min at room temperature. 7. Spin down crosslinked cells at 1000×g for 5 min at 4 degrees C. 8. Discard quenched media in labeled waste bottle and wash cells twice with 25 mls chilled PBS. 9. After the last wash, add 1 ml chilled PBS. 10. Count cell numbers; then aliquot 0.5-3 million cells per tube. 11. Spin at 1000×g for 5 minutes at 4° C. and discard 1×PBS buffer. 12. Freeze pellets. The cell pellets can be frozen at −80° C. for up to six months.

### D1 Cell Lysis and Sonication

### Cell Lysis

Prepare EB buffer and FA lysis buffer containing 1× proteinase inhibitor. 1. Thaw cells on the ice for 5-10 min. 2. Resuspend cells in 1 ml of 0.1% FA Lysis buffer with 1× protease inhibitor. 3. Rotate at 4° C. for 10 min (20 rpm). 4. Spin at 1700×g for 5 min at 4° C. Discard the supernatant. 5. Resuspend cells in 1 ml of 0.1% FA Lysis buffer with 1× protease inhibitor. 6. Rotate at 4° C. for 20 min (20 rpm). 7. Spin at 1700×g for 5 min at 4° C. Discard the supernatant. 8. Resuspend cells in 1 ml of 1% FA nuclear Lysis buffer with 1× protease inhibitor. 9. Rotate at 4° C. for 15 min (20 rpm). 10. Spin at 1700×g for 5 min at 4° C. Discard the supernatant; then, repeat 8-10 once. 11. Resuspend pellet with 1 ml cold EB buffer, mix well, Spin at 1700×g for 5 min at 4° C. Discard the supernatant; then, repeat step 11 once. In step 11, the pellet appears puffy and transparent in the final step, and is enough to cover the bottom of the tube. If it's not enough, increase the number of cells for lysis and combine them in the next step. Steps 8-11 permeabilize nuclear to make it easier to be sheared in the next steps. EB buffer wash step is for buffer exchange purpose.

### Sonication

1. Resuspend pellet with 130 μl EB buffer. 2. Keeping the cap on the COVARIS® tubes (PN 520045), using a tapered pipette tip, transfer 130 μl of DNA sample (in EB buffer) by inserting the pipette tip through the pre-split septa. 3. Place COVARIS® tubes on “Rack 24 Place microTUBE Snap-Cap (PN 500111)”. NOTE: The water level should be ~6. 4. Setup parameters then start sonication once de-gas is done. Sonication parameters (COVARIS® E220 with intensifier): Peak Incident=105; Duty Factor=2%, Cycles per Burst=200; Treatment Time of 4 min; Temperature of 4-9° C. Set treatment time to 2, 4, 6, 8, 10, and 12 min for optimizing fragment size. 5. Open the cap of the COVARIS® tube, transfer ~130 μl sonicated chromatin from tube to a new 1.5 ml microtube. If needed, increase the concentration of chromatin by combining multiple samples (e.g. 130+130 μl) if enough pellet is not obtained during the lysis step. Adjust the volume of EB buffer to reach a total of 500 μl for step 6. 6. Add 370 μl EB buffer containing 1× protease inhibitor to make it 500 μl. 7. Spin at full speed and 4° C. for 10 min. 8. Carefully transfer the supernatant to new 1.5 ml microtube. 9. Take 15 μl (3%) of chromatin for QC.

### Sonication QC (QC1)

1. Take 15 μl (~3%) of sonicated chromatin for QC sonication, and mix with 80 μl TE and 5 μl Proteinase K. Incubate in the ThermoMixer at 65° C., 900 rpm for 30-60 min. 2. Purify the sample using Zymo ChIP DNA Clean and Concentrator by following manufacturer's manual, eluted in 30 μl elution buffer. 3. Take 1 μl for analyzing fragment size by TapeStation Genomic DNA. 4. Take 1 μl for measuring concentration by Qubit dsDNA HS. Both yield and size should be similar to QC1. To be qualified, the total yield of chromatin should be at least 10 μg, where more than 90% of fragments are under 7000 bp, over 50% of fragments are between 1000-7000 bp, and no significant peak around 500 bp.

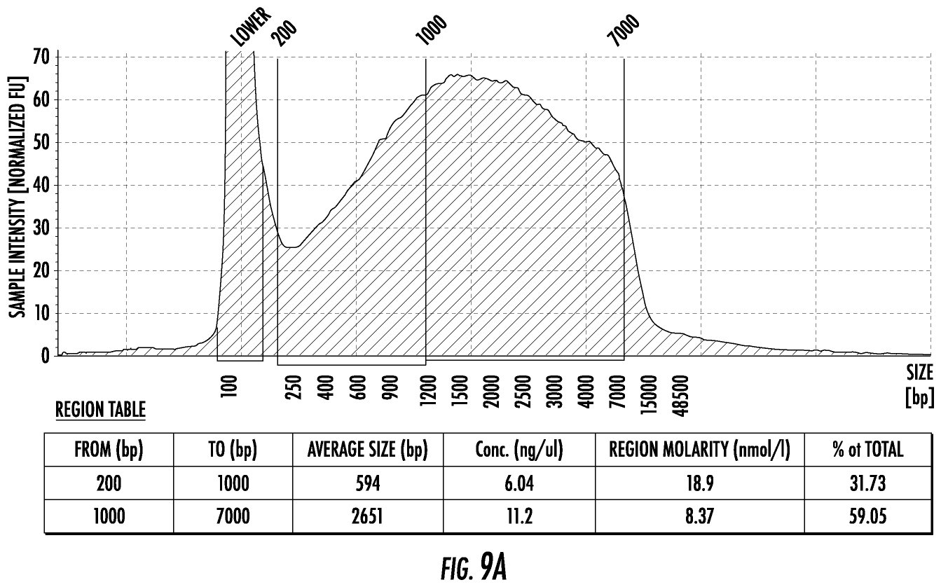

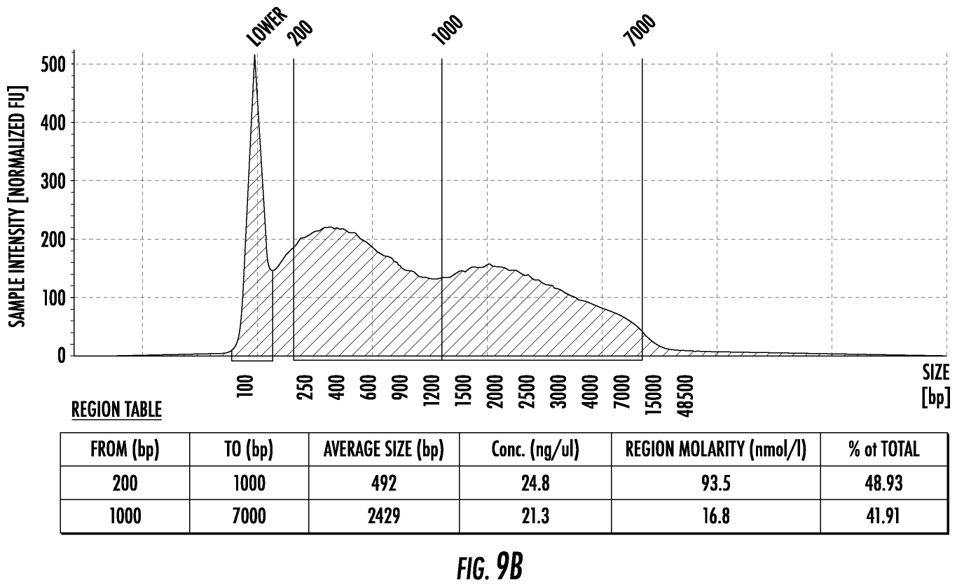

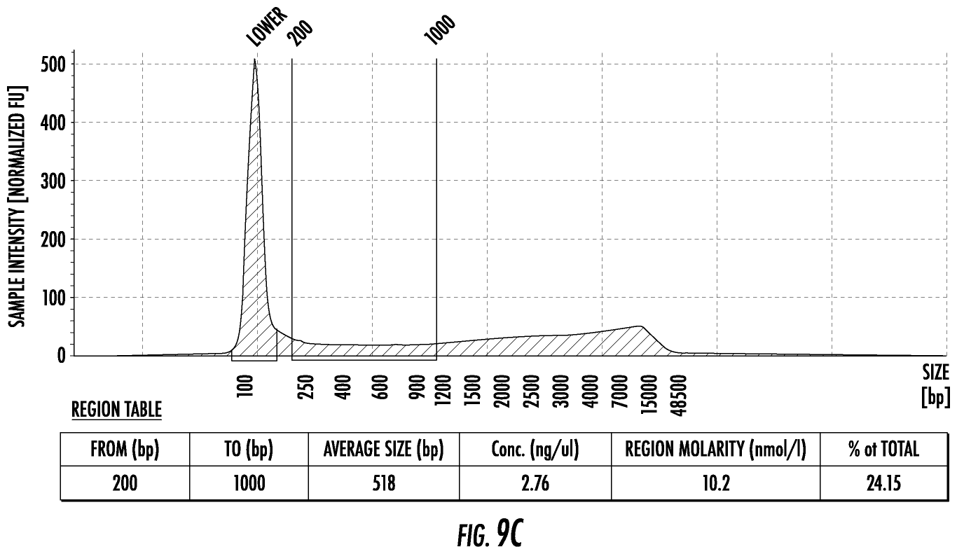

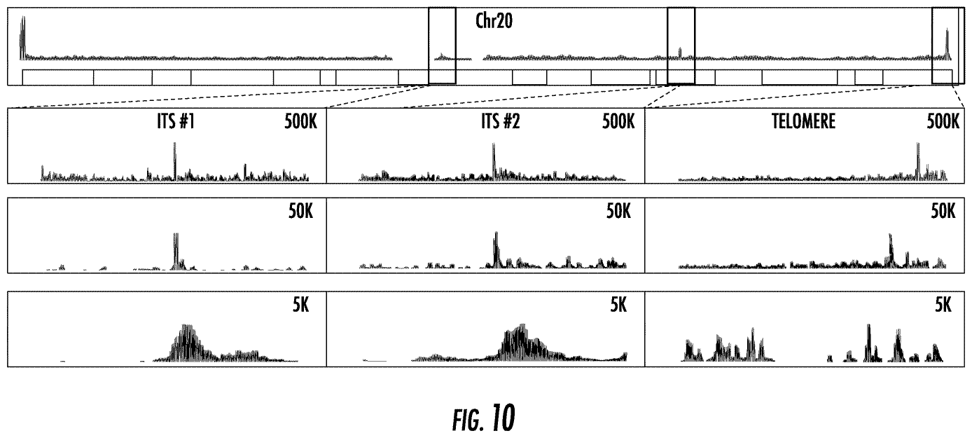

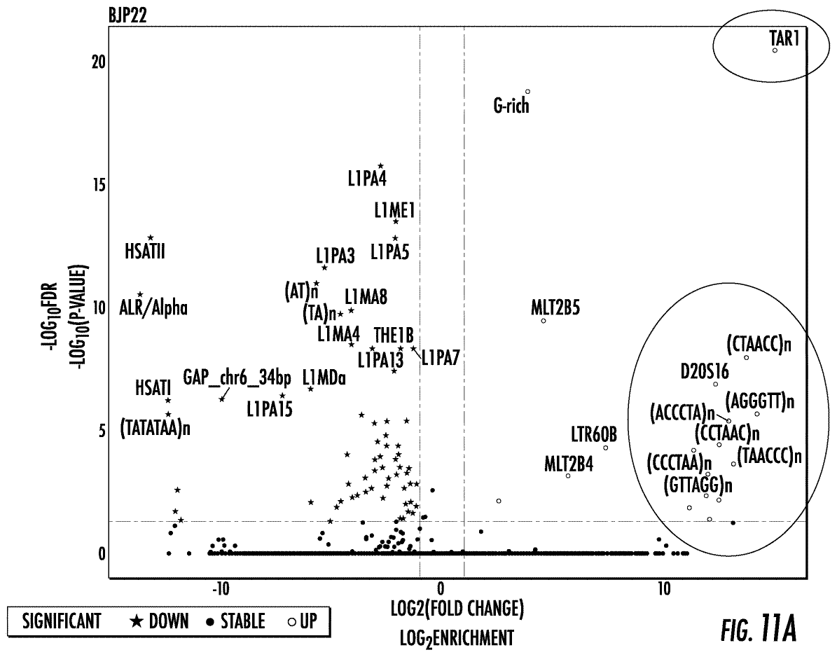

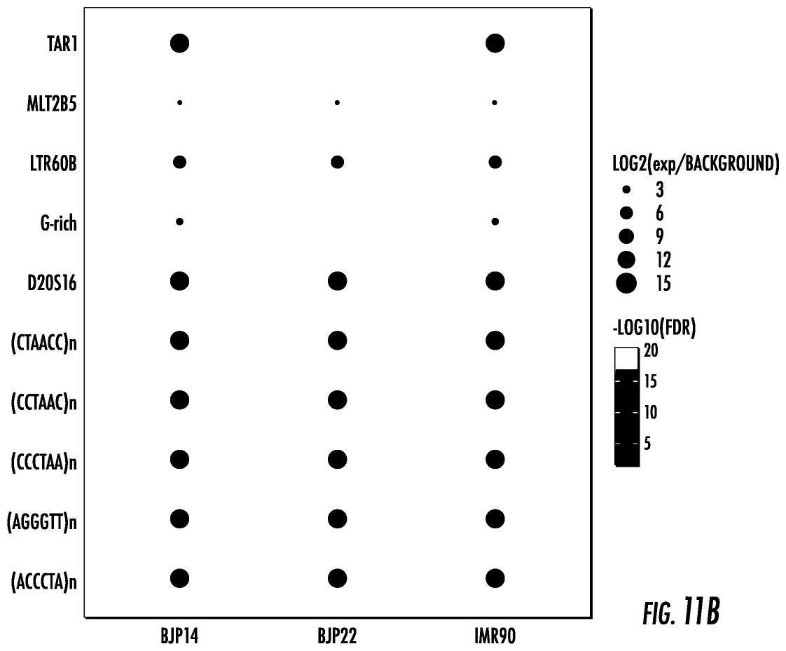

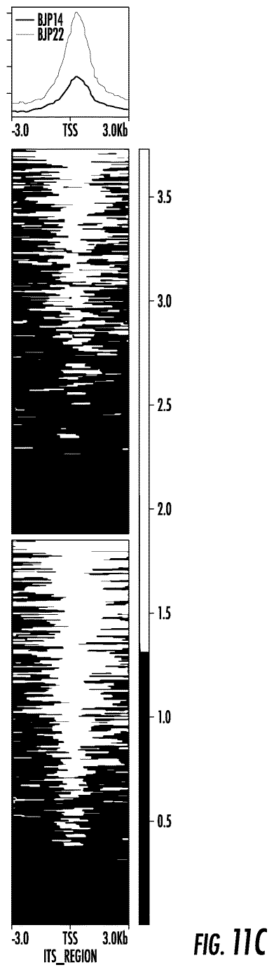

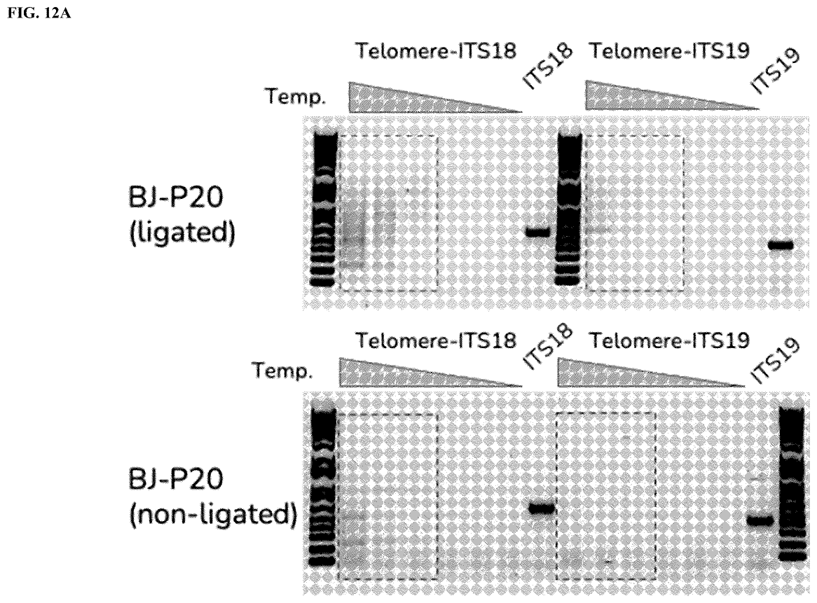

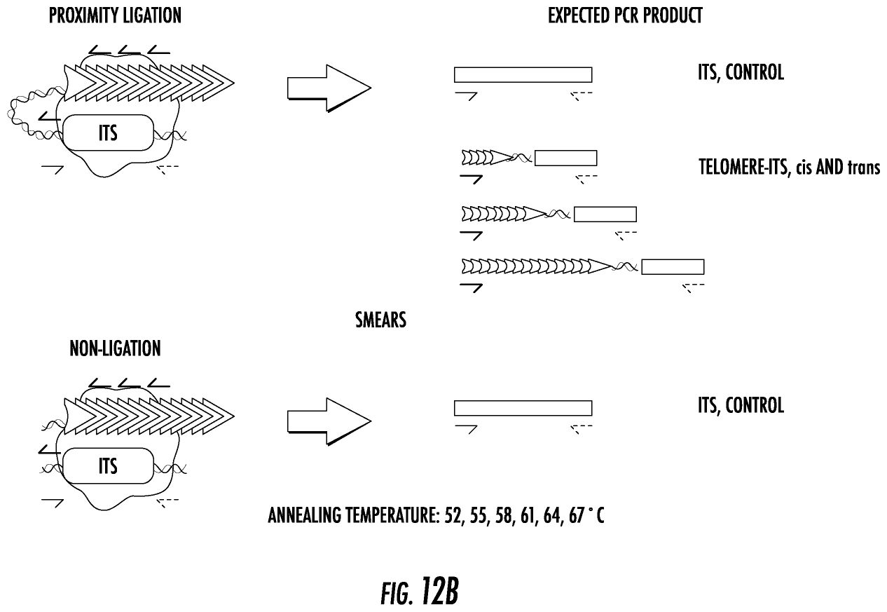

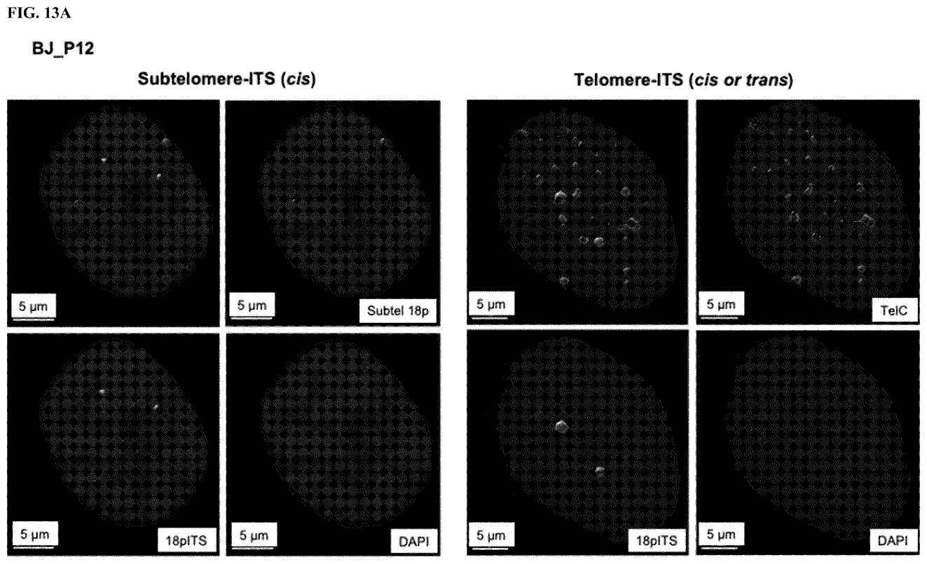

FIGS. 9A, 9B and 9C show examples fragment yield and size quality. FIG. 9A shows good quality. FIG. 9B shows over shearing. FIG. 9C shows under shearing.

### Chromatin Concentration

1. Carefully transfer the supernatant to Amicon Ultra-0.5 ml (Ultracel-100K) column. spin at 4° C. 14000×g for 10 min, discard the bottom part. 2. Invert the column to another clean 2 ml collection tube, spin at 4° C. 1000×g for 2 min, to get the concentrated chromatin fragments (~50 μl). 3. This filtered chromatin can be stored at 4° C. overnight.

### D2 Chromatin Pre-Clearing

Shake the Dynabeads MyOne T1 bottle to resuspend any magnetic particles that may have settled. Equilibrate to room temperature. Prepare LBJD, LBJDLS buffer containing 1× protease inhibitor.

### Dynabeads Pre-Equilibration

1. For each reaction, transfer 50 μl Dynabeads MyOne T1 into 1.5 microtubes. 2. Add 1 ml 1×LBJDLS buffer and resuspend the beads. 3. Keep the lid of the microtube open, and place the tube on a magnetic stand for 2 min and discard the supernatant. 4. Resuspend beads in 450 μl LBJDLS buffer. 5. Add beads to 50 μl chromatin. 6. Rotate at 4° C. overnight for 2 hour (20 rpm). 7. Keep the lid of the microtube open, and place the tube on a magnetic stand for 2 min then transfer the supernatant to a new 1.5 ml microtube.

### Input Chromatin QC (QC2)

1. Take 15 μl (~3%) of pre-cleared chromatin for QC, and mix with 80 μl TE and 5 μl Proteinase K. Incubate in the ThermoMixer at 65° C., 900 rpm for 30-60 min. 2. Purify the sample using Zymo ChIP DNA Clean and Concentrator by following manufacturer's manual, eluted in 30 μl elution buffer. 3. Take 1 μl for measuring concentration by Qubit dsDNA HS. 4. Save DNA for the input control.

### Hybridization

Having no probe negative control is recommended to evaluate capture efficiency. Expected to get 1%-0.1% of input.

1. Add 5 μl PNA (25 μM) to a final concentration of 0.25 μM. For no probe control, add 5 μl water. 2. Dispense 100 μl mixture into 4-5 PCR tubes. 3. Hybridize in a standard thermocycler using the following program: (a) 25° C. for 3 min; (b) 71° C. for 9 min; (c) 38° C. for 1 hour; (d) 25° C. final temperature. 4. Transfer the samples from the PCR tubes into 1.5 mL microtubes and centrifuge at 16,000×g for 15 min at room temperature in order to pellet any precipitate that could have formed during the hybridization step. 5. Transfer the supernatants into 1.5 mL microtubes. 6. This captured chromatin can be stored at 4° C. overnight.

### D3 Chromatin Capture

Shake the Dynabeads MyOne T1 bottle to resuspend any magnetic particles that may have settled. Equilibrate to room temperature.

### Capture

1. For each reaction, transfer 100 μl Dynabeads MyOne T1 into 1.5 ml microtubes. 2. Add 1 ml 1×LBJDLS buffer and resuspend the beads. 3. Keep the lid of the microtube open, and place the tube on a magnetic stand for 2 min and discard the supernatant. 4. Repeat step 2-3. 5. Keep the lid of the microtube open, and place the tube on a magnetic stand for 2 min and discard the supernatant. 6. Keep the beads on the stand while adding 500 μl chromatin. 7. Still keeping beads on the stand dilute the chromatin in 500 μl of Milli-Q water. 8. Resuspend the beads very gently and slowly. Incubate for 90 min on a rotator at room temperature.

### Wash

1. Wash the beads five times with 1 ml LBJD buffer. Resuspend the beads gently between the washes, avoid vortexing. Perform the 2 times wash with 1 ml LBJDLS buffer. 2. Resuspend in 1 mL of LBJDLS buffer. 3. Incubate for 5 min at 42° C. in a thermomixer shaking at 500 rpm. 4. Immobilize the beads on the magnetic stand and discard the supernatant. 5. Resuspend in 700 TE buffer. During wash steps, the beads that capture probe-chromatin may require more effort to resuspend compared to the no-probe control beads, which are easier to suspend.

### Decrosslinking

1. Add 25 μl Proteinase K (10 mg/ml) to 700 μl ligation product, mix by pipetting; then place in the ThermoMixer at 65° C. for 3 h 30 min-4 h, 900 rpm. 2. Keep the lid of the microtubule open, and place the tube on a magnetic stand for 2 min. 3. Transfer 725 μl decrosslinked product to 2 ml microtube. 4. This product can be stored at 4° C. overnight.

### D4 Phenol-Chloroform Purification

Adjust phenol-chloroform to pH 7.9. 1. To settle gel to bottom, centrifuge 1.5 ml MaXtract high density tubes at full speed, room temperature for 2 minutes. 2. Add 725 μl Phenol-Chloroform-Isoamyl alcohol (pH 7.9) to 725 μl decrosslinked proximity ligated product. 3. Vortex tube vigorously for 10 seconds or until it turns white; then transfer the entire volume to the MaXtract tube. 4. Centrifuge the MaXtract tube at full speed, room temperature for 5 minutes. 5. Carefully collect the upper ~577.5 μl aqueous layer to a new 2 ml microtube. 6. Add the following components into the aqueous solution in the 2 ml microtube by following order: 72.5 μl 3M Sodium Acetate pH 5.5; 2.5 μl GlycoBlue; 797.5 μl Ice-cold Isopropanol. 7. Invert tube to mix well, incubate at −20° C. for 15 minutes. 8. Centrifuge at full speed, 4° C. for 20 minutes to precipitate DNA; then, aspire supernatant. 9. Add 1 ml ice-cold 75% ethanol to wash the pellet gently, avoid breaking the pellet. The pellet is smaller for the no-probe control, whereas for captured chromatin, the pellet appears rough with white edges. 10. Centrifuge at full speed, 4° C. for 5 minutes; then aspire supernatant. 11. Repeat step 9-10 two times. 12. Keep the lid of the microtube open, place the tube in the ThermoMixer at 37° C. for (5-10 min) to dry the pellet. 13. Resuspend pellets in 10 μl Qiagen EB buffer, then transfer to 1.5 microtube.

### Capture QC (QC3)

Take 2 μl to DNA concentration by Qubit as QC3. The yield of capture DNA should be 1%-0.1% of input. While, <0.1% or undetectable DNA in the no probe control.

### Secondary Digestion AluI

Perform AluI digestion in the End-repairing and A-tailing buffer

| | Input | | Capture | |
|---|---|---|---|---|
| DNA | X μl (5-50 ng) | | X μl (5-50 ng) | |
| ER-A buffer | 3.5 | μl | 3.5 | μl |
| AluI | 1 | μl | 1 | μl |
| H2O | Y | μl | Y | μl |
| total | 30 | μl | 30 | μl |

Incubate at 37° C. for 1hr, then 80° C. for 20 min

| DNA | Passed from previous step | | Passed from previous step | |
|---|---|---|---|---|
| ER-Abuffer | 3.5 | μl | 3.5 | μl |
| ER-A enzyme | 3 | μl | 3 | μl |
| H2O | 23.5 | μl | 23.5 | μl |
| Total | 60 | μl | 60 | μl |

### ILLUMINA® Adaptor Ligation

### Library End Repair+A-Tailing

1. Add additional buffer and ER-A enzyme to perform End Repair & A-Tailing.

| DNA | Passed from previous step | |
|---|---|---|
| ER-Abuffer | 3.5 | μl |
| ER-A enzyme | 3 | μl |
| H2O | 23.5 | μl |
| Total | 60 | μl |

2. Place tubes on thermocycler, and set program by following:

| | | |
|---|---|---|
| Steps | Temperature | Time |

| | | | |
|---|---|---|---|
| 1 | 20° | C. | 30 min |
| 2 | 65° | C. | 30 min |
| 3 | 4° | C. | hold |

### Library Adaptor Ligation

1. Prepare ligation master mix for each reaction: 30 μl KAPA ligation buffer, 10 μl KAPA DNA ligase, 5 μl 15 μM IDT TruSeq Adaptor, 5 μl Nuclease-free water. Note: Dilute adaptor according to bellowing table if input concentration≤25 ng. 2. Mix 50 μl ligation master mix with 60 μl of A-tailed product in a 1.5 ml microtube. 3. Incubate the tube in the ThermoMixer at 25° C. for 25 minutes.

TABLE 2
Recommended adapter concentrations for libraries constructed from 1 ng-1 μg input DNA*

| Fragmented DNA | | | | Fragmented DNA | | | | |
|---|---|---|---|---|---|---|---|---|
| per 50 μL | | Adapter stock | Adapter:insert | per 50 μL | | Adapter stock | | Adapter:insert |
| ER & AT reaction | | concentration | molar ratio | ER & AT reaction | | concentration | | molar ratio |
| 1 | μg | 15 μM | 10:1 | 25 | ng | 7.5 | μM | 200:1 |
| 500 | ng | 15 μM | 20:1 | 10 | ng | 3 | μM | 200:1 |
| 250 | ng | 15 μM | 40:1 | 5 | ng | 1.5 | μM | 200:1 |
| 100 | ng | 15 μM | 100:1 | 2.5 | ng | 750 | nM | 200:1 |
| 50 | ng | 15 μM | 200:1 | 1 | ng | 300 | nM | 200:1 |

*Adapter:insert molar ratio calculations are based on a mode DNA fragment length of 200 bp, and will be higher for longer DNA fragments, or slightly lower for DNA fragmented to a mode size <200 bp. The lower adapter:insert molar ratios recommended for inputs >100 ng represent a fair compromise between library construction efficiency and cost; higher library yields will be achieved if a higher adapter concentration is used.

### Clean Up, 1.8× AMPure XP

Prepare 10 ml fresh made 80% EtOH (for 8 rxn). 1. Add 198 μl volume of Ampure XP beads into 110 μl product. Mix by pipetting 10 times. 2. Incubate at room temperature for 5 min. 3. Keep the lid open then place the microtube on the magnet. Stand for 2 minutes to separate beads from the solution. 4. Aspirate the cleared solution from the reaction plate and discard. 5 Dispense 300 μL of 80% ethanol to each well of the reaction plate and incubate for 30 seconds at room temperature. Aspirate out the ethanol and discard. Repeat for a total of two washes. 6. Take the microtube from the magnetic stand, close the lid, and quickly spin to settle beads to the bottom of the tube. 7. Keep the lid of the microtube open, place the tube in the magnetic stand. 8. Aspirate residual EtOH on the bottom of the microtube by p10 pipetman. 9. Air dry the pellet for 5-10 min at room temperature. 10. Resuspend pellet in 53 μl Qiagen EB buffer. 11. Incubate at room temperature for 5 min. 12. Keep the lid of the microtube open then place the tube on the magnetic stand for 2 min. 13. Transfer 50 μl supernatant to a new 1.5 microtube.

### Size Selection, 0.8× (L-Side)

Prepare 10 ml fresh made 80% EtOH (for 8 rxn). 1. Add 40 μl volume of Ampure XP beads into 50 μl cleaned product. Mix by pipetting 10 times. 2. Incubate at room temperature for 5 min. 3. Keep the lid open then place the microtube on the magnet stand for 2 minutes to separate beads from the solution. 4. Aspirate the cleared solution from the reaction plate and discard. 5. Dispense 125 μL of 80% ethanol to each well of the reaction plate and incubate for 30 seconds at room temperature. Aspirate out the ethanol and discard. Repeat for a total of two washes. 6. Take the microtube from the magnetic stand, close the lid, and quickly spin to settle beads to the bottom of the tube. 7. Keep the lid of the microtube open, place the tube in the magnetic stand. 8. Aspire residual EtOH on the bottom of the microtube by p10 pipetman. 9. Air dry the pellet for 5-10 min at room temperature. 10. Resuspend pellet in 43 μl Qiagen EB buffer. 11. Incubate at room temperature for 5 min. 12. Keep the lid of the microtube open then place the tube on the magnetic stand for 2 min. 13. Transfer 40 μl supernatant to a new 1.5 microtube. 14. Sample can be stored at −20° C.

### Library Amplification

1. Set up PCR reaction.

| Compnet | Amount per rxn |
|---|---|
| 2X KAPA HiFi HotStart ReadyMix | 50 μl |
| 20 μM P5/P7 primers | 10 μl |
| Library | 40 μl |

2. Perform PCR.

| | | |
|---|---|---|
| Steps | Temperature | Time |

| | | | | |
|---|---|---|---|---|
| 1 | 98° | C. | 45 | sec. |
| 2 (N cycles) | 98° | C. | 15 | sec. |
| | 60° | C. | 30 | sec. |
| | 72° | C. | 30 | sec. |
| 3 | 72° | C. | 1 | min. |

| | | | |
|---|---|---|---|
| 4 | 4° | C. | Hold |

TABLE 3
Recommended cycle numbers to generate 100 ng or 1 μg of
amplified DNA from 1 ng-1 μg of input DNA

| | |
|---|---|
| Input DNA | Number of cycles required to generate |

| | | |
|---|---|---|
| (into ER and AT) | 100 ng library | 1 μg library |

| | | | |
|---|---|---|---|
| 1 | μg | 0* | 1-2* |
| 500 | ng | 0* | 2-4 |
| 250 | ng | 0-2* | 4-6 |
| 100 | ng | 2-3* | 6-7 |
| 50 | ng | 3-5 | 7-8 |
| 25 | ng | 5-7 | 8-10 |
| 10 | ng | 7-9 | 11-13 |
| 5 | ng | 9-11 | 13-14 |
| 2.5 | ng | 11-13 | 14-16 |
| 1 | ng | 13-15 | 17-19 |

*When using incomplete adapters, a minimum number of amplification cycles (1-3) may be required to complete adapter sequences for the next step in the process (target capture or sequencing), irrespective of whether a sufficient amount of library is available after ligation. The number of cycles needed depends on the specific adapter and amplification primer design.

### Clean Up, 1× AMPure XP

1. Add 100 μl volume of Ampure XP beads into 100 μl product. Mix by pipetting 10 times. 2. Incubate at room temperature for 5 min. 3. Keep the lid open then place the microtube on the magnet. Stand for 2 minutes to separate beads from the solution. 4. Aspirate the cleared solution from the reaction plate and discard. 5. Dispense 200 μL of 80% ethanol to each well of the reaction plate and incubate for 30 seconds at room temperature. Aspirate out the ethanol and discard. Repeat for a total of two washes. 6. Take the microtube from the magnetic stand, close the lid, and quickly spin to settle beads to the bottom of the tube. 7. Keep the lid of the microtube open, place the tube in the magnetic stand. 8. Aspirate residual EtOH on the bottom of the microtube by p10 pipetman. 9. Air dry the pellet for 5-10 min at room temperature. 10. Resuspend pellet in 53 μl Qiagen EB buffer. 11. Incubate at room temperature for 5 min. 12. Keep the lid of the microtube open then place the tube on the magnetic stand for 2 min. 13. Transfer 50 μl supernatant to a new 1.5 microtube.

### Double Size Selection, 0.8× (L-side)

1. Add 40 μl volume of Ampure XP beads into 50 μl cleaned product. Mix by pipetting 10 times. 2. Incubate at room temperature for 5 min. 3. Keep the lid open then place the microtube on the magnet stand for 2 minutes to separate beads from the solution. 4. Aspirate the cleared solution from the reaction plate and discard. 5. Dispense 125 μL of 80% ethanol to each well of the reaction plate and incubate for 30 seconds at room temperature. Aspirate out the ethanol and discard. Repeat for a total of two washes. 6. Take the microtube from the magnetic stand, close the lid, and quickly spin to settle beads to the bottom of the tube. 7. Keep the lid of the microtube open, place the tube in the magnetic stand. 8. Aspire residual EtOH on the bottom of the microtube by p10 pipetman. 9. Air dry the pellet for 5-10 min at room temperature. 10. Resuspend pellet in 53 μl Qiagen EB buffer. 11. Incubate at room temperature for 5 min. 12. Keep the lid of the microtube open then place the tube on the magnetic stand for 2 min. 13. Transfer 50 μl supernatant to a new 1.5 microtube. 14. Sample can be stored at −20° C.

### Double Size Selection, 0.56× (R-side)

1. Add 28 μl (0.56×) volume of Ampure XP beads into 50 μl library. Mix by pipetting 10 times. 2. Incubate at room temperature for 5 min. 3. Keep the lid open then place the microtube on the magnet stand for 2 minutes to separate beads from the solution.

4. Transfer supernatant to clear 1.5 ml microtube. 5. Add 62 μl (1.8×-0.6×) of Ampure XP beads into the supernatant from 4. Mix by pipetting 10 times. 6. Incubate at room temperature for 5 min. 7. Keep the lid open then place the microtube on the magnet stand for 2 minutes to separate beads from the solution. 8. Aspirate the cleared solution. 9. Dispense 200 μL of 80% ethanol to each well of the reaction plate and incubate for 30 seconds at room temperature. Aspirate out the ethanol and discard. Repeat for a total of two washes. 10. Take the microtube from the magnetic stand, close the lid, and quickly spin to settle beads to the bottom of the tube. 11. Keep the lid of the microtube open, place the tube in the magnetic stand. 12. Aspire residual EtOH on the bottom of the microtube by p10 pipetman. 13. Air dry the pellet for 5-10 min at room temperature. 14. Resuspend pellet in 53 μl Qiagen EB buffer. 15. Incubate at room temperature for 5 min. 16. Keep the lid of the microtube open then place the tube on the magnetic stand for 2 min. 17. Transfer 50 μl supernatant to a new 1.5 microtube. 18. Take 2 μl of the library for analyzing fragment size by TapeStation HSD5000. 19. Take 1 μl of the library for qPCR the quantify concentration.

### Results

The following results show that the ligation-free chromatin capture and sequencing method produces a resulting signal (referred to herein as “Telomere-C signal”) that is strongest near chromosome ends and gradually declines over the length of a chromosome arm. The resulting signal shows a striking periodicity, suggestive of regularly spaced chromatin loops. The Telomere-C signal is markedly pronounced at sites of interstitial telomeric sequences (ITS), potentially representing chromatin hubs of multiple interacting telomeric loci. A peak was found in the vicinity of the TERT locus, which could point to a telomere-length dependent auto-feedback loop controlling TERT transcription.

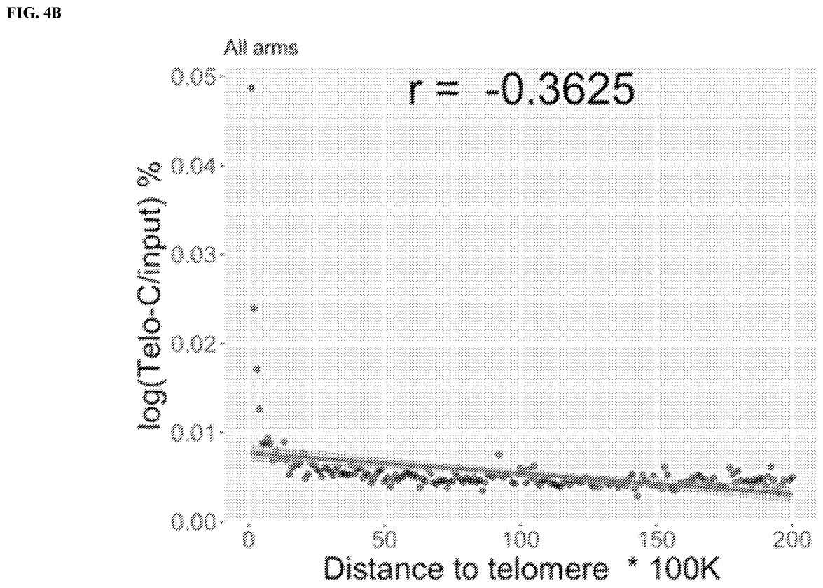

FIGS. 4A and 4B show the negative association between resulting signals and chromosome ends. FIG. 4B shows the gradual decline of Telomere-C signal along chromosome arms, indicating that Telomere-chromatin interactions are rich at the end of chromosomes. FIG. 4A shows Telomere-C signal (purple bar) and input (blue bar) were visualized using IGV in cell lines at the end of Chr19q13 (CHM13v2). FIG. 4B shows the average Telomere-C signal within 20 MB of the p and q arms was calculated, normalized with input, and displayed in a log scale. The r value of the Pearson correlation and the trend line from the general linear regression model (blue line) are shown.

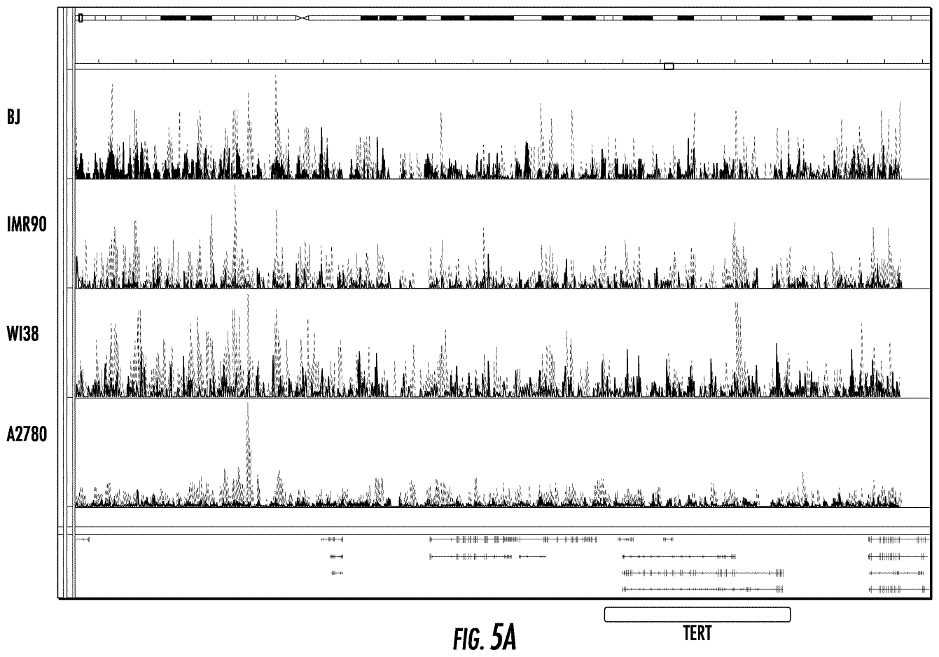

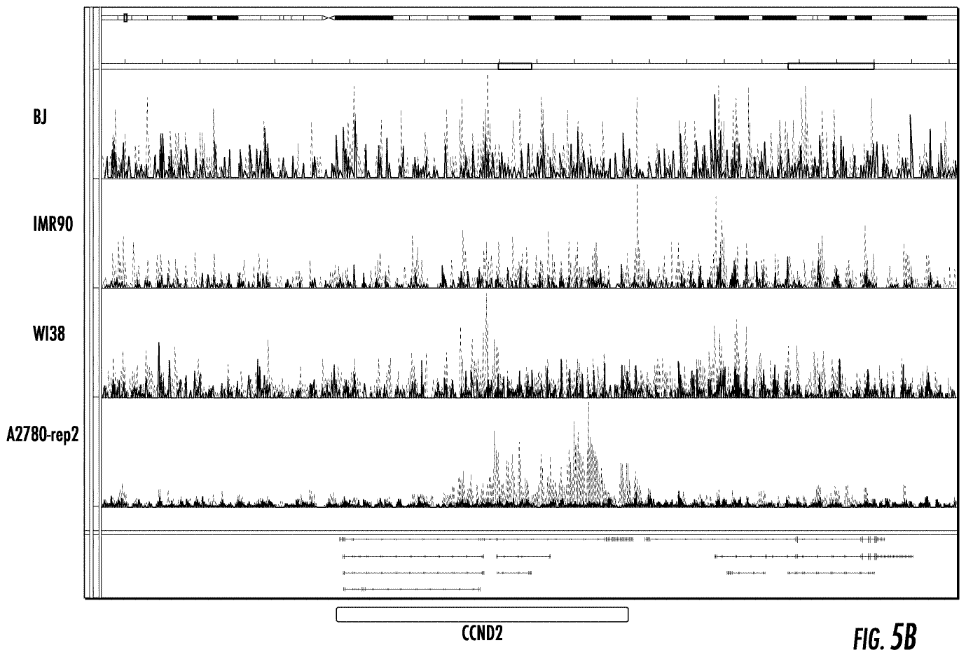

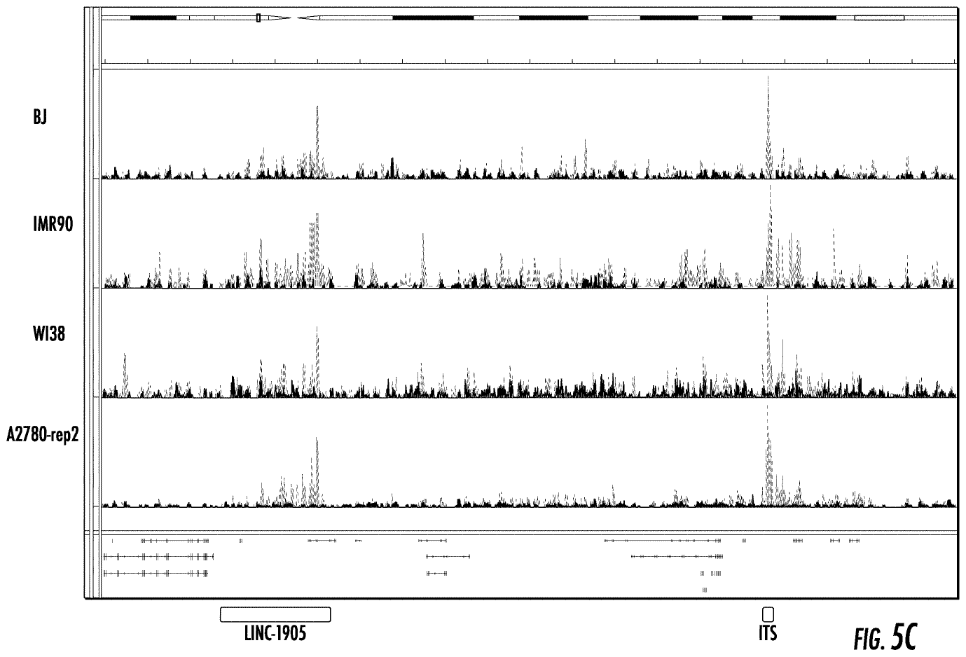

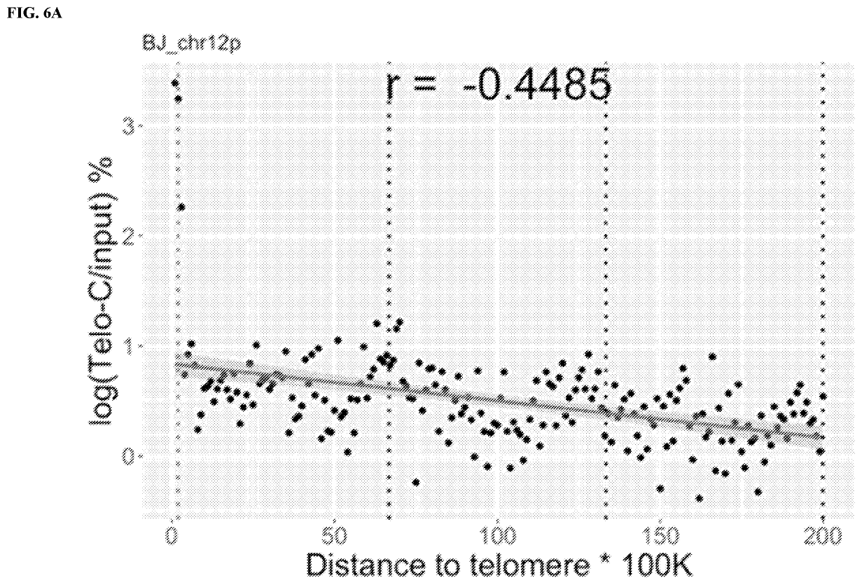

FIGS. 5A, 5B, and 5C show telomeric chromatin interacts in the loci of TERT, CCND2, ITS, and at consensus sites and show cell-specific and consensus Telomere-C signals at different genomic Loc. Telomere-C signal (purple bar) and input (blue bar) were visualized using IGV in the indicated cell lines at the loci of TERT (FIG. 5A), CCND 2 (FIG. 5B), LINC-1905 (FIG. 5C), and ITS. Cell-specific signals were observed in TERT and CCND2, while consensus signals were observed in LINC1905 and ITS.

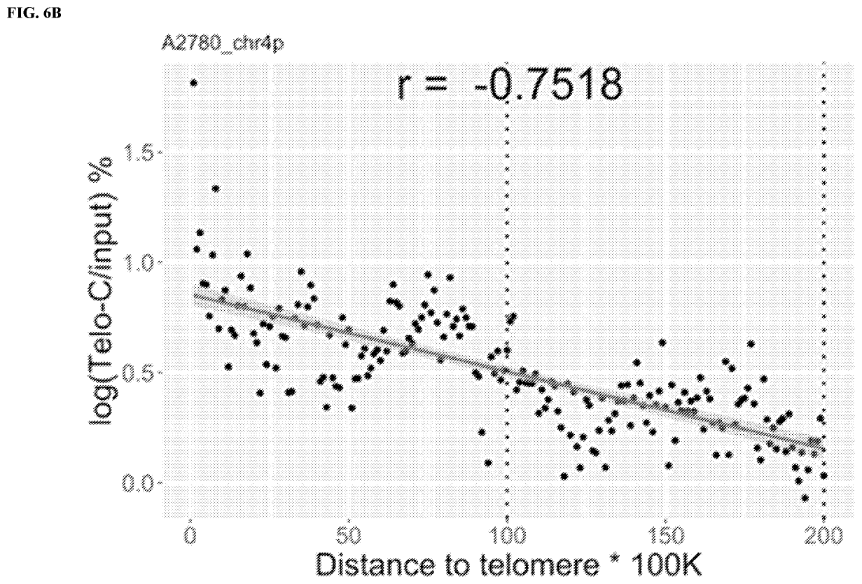

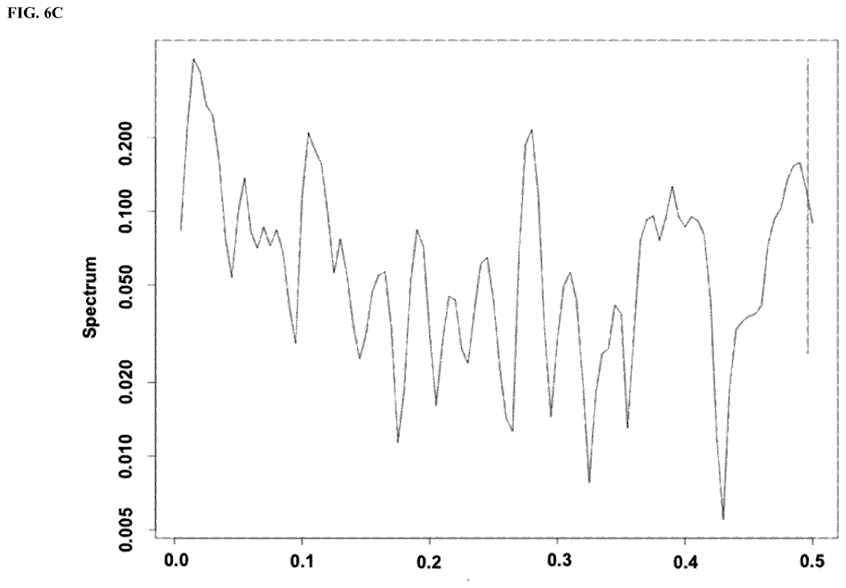

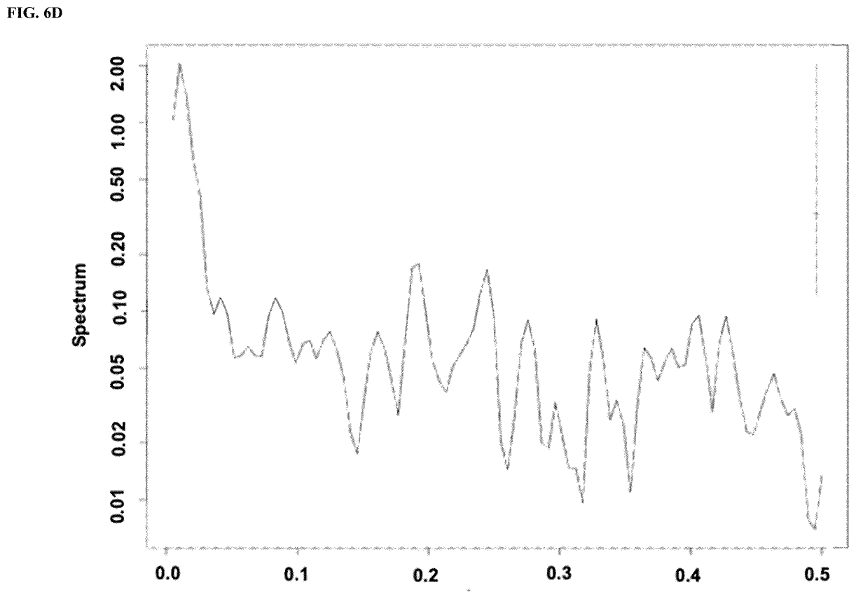

FIGS. 6A-6D show the periodicity of Telomere-C signals at the end of chromosomes. FIGS. 6A and 6B show Telomere-C signal within 20 MB of the p arm in BJ (FIG. 6A) or (in FIG. 6B) A2780 cells, calculated and normalized with input and displayed in a log scale. The Pearson correlation (r value) and trend line from the general linear regression model (blue line) are shown. The black dotted line indicates the interval of the period, and the red dotted line indicates the bin of ITS. FIGS. 6C and 6D display periodograms, showing the dominant and minor frequency of Telomere-C signal. The periodicity of the Telomere-C signal suggests long-distance telomere-chromatin interactions.

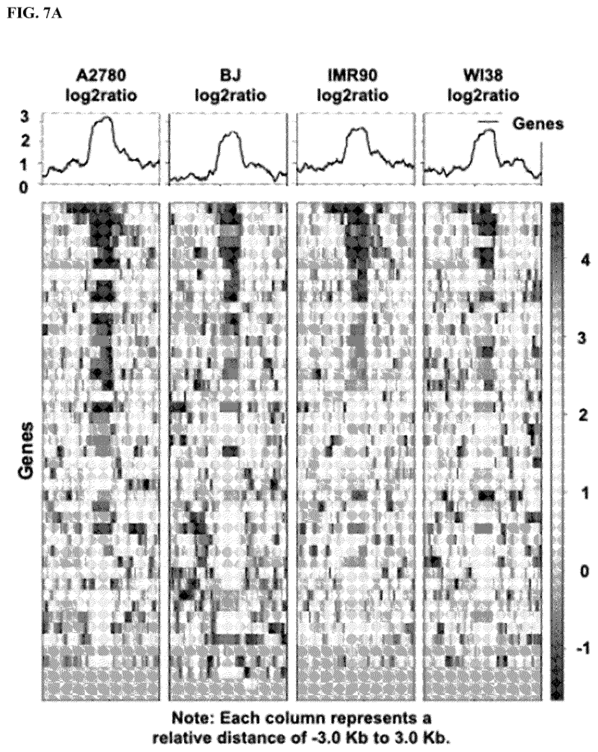

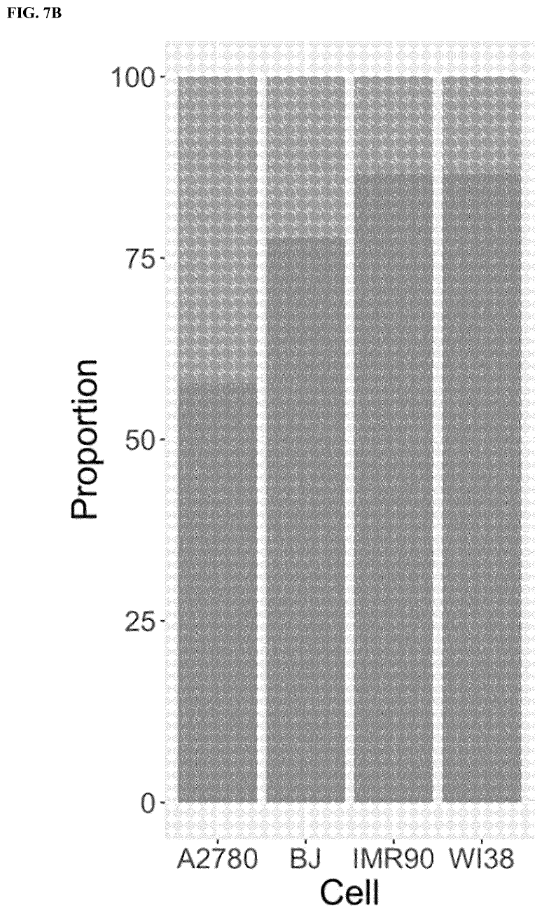

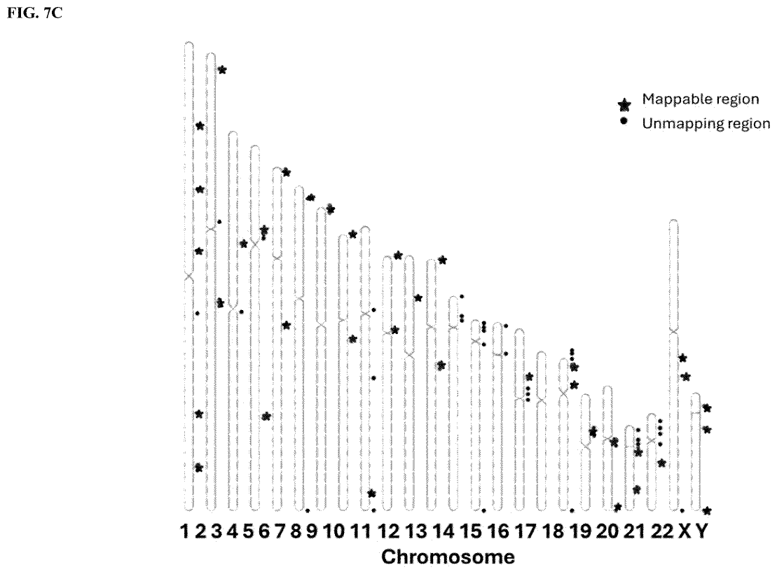

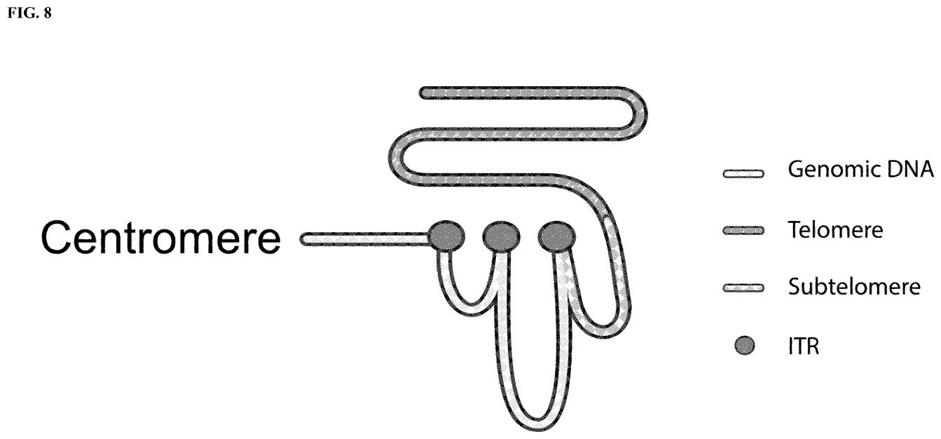

FIGS. 7A, 7B, and 7C show enrichment of Telomere-C signals in ITS regions. FIG. 7A shows a heatmap showing Telomere-C signals in the ITS region and its upstream and downstream regions in A2780, BJ, IMR90, and WI38 cells. Normalized signals are displayed in a line chart in the top panel and in the heatmap, with weaker signals shown in red and stronger signals shown in blue. FIG. 7B is a Bar chart showing the proportion of ITS regions (n=45) that overlap with MACS2-called Telomere-C peaks (red bar). FIG. 7C is an ideogram to show the distribution of ITS in 45 mappable (blue) or 33 unmappable regions (black). Telomere-C signal is markedly pronounced at interstitial telomeric sequences (ITS) sites, indicating that ITS serves as a hub of telomere-chromatin interaction.

These preliminary results in a human ovarian cancer cell line, A2780, show that the signals produced with the ligation-free chromatin capture and sequencing method are negatively associated with the distance from telomeres and strongly associated with interstitial telomere sequence (ITS), indicating remote interaction sites. An association of the resulting signals was observed with part of CTCF or H3K27ac peaks, suggesting a relationship between telomeric chromatin interaction and epigenetic regulation of transcription. In addition, a bundle of the resulting signals was found in the locus (DUX4, CCND2), upstream of (ISG15), and downstream of (TERT) genes. These gene expressions are regulated by TPE-OLD or dependent on telomere length, as indicated by recent literature.

These findings provide insights into the chromatin interactions occurring at telomeres and their potential role in aging and age-related diseases by allowing investigation into the higher-order chromatin structure of telomeres and their relationship to the rest of the genome. For example, the disclosed approach will benefit the study of age-related diseases, such as cancer and Alzheimer's disease, by untangling how changing telomeric chromatin interactions influence cellular phenotypes.

Further Analysis with Telomere-C, 3C, and FISH

Detection of proximal and distal telomere-chromatin interaction in Chr20 by Telomere-C is shown in FIG. 10. The signal of Telomere-C has been plotted in a chromosome-wide view. Strong signals of telomere-chromatin interaction in the terminal regions of chromosomes (proximal), regions of interstitial telomere sequences (ITSs), or centromeric ITSs (distal) in chromosome 20 can be easily visualized in these representative results.

In FIGS. 11A-11C, the richness of Telomere-C signals in the Telomere Associated Repeat 1, (CCCTAA)n, or (TTAGGG)n, and other repeat elements is shown. The signal of Telomere-C in the regions of repeat elements (RepeatMasker v4.1.2p1.2022Apr14) has been analyzed by comparing the signals between repeat regions and random regions. It has been found that Telomere-C is rich in the non-telomeric (TTAGGG)n or (CCCTAA)n regions, which are potential regions of ITS. Additionally, Telomere-C signals have been found to be significantly rich in repeat elements that don't contain TTAGGG (or CCCTAA) sequences such as telomere-associated repeats (TAR1) or D20S16. The same discovery has been found in early passages and later passages of BJ cells (BJP14 and BJP22), and IMR90 cells.

Validation of Telomere-ITSs interaction by 3C is shown in FIGS. 12A and 12B. Chromatin conformation capture (3C) was performed to detect the interaction between telomere and selected ITSs on chromosome 18 or chromosome 19. Chromatin was crosslinked with 1.5 mM EGS and 1% formaldehyde followed by enzymatic digestion with Alu I. Proximity ligation with T4 ligase (or without T4 ligase as negative control) was conducted, and reverse crosslinking was performed with proteinase K. The 3C DNA was purified applying the phenol-chloroform extraction method for the PCR reaction with customized primers. Electrophoresis in 1% agarose gel was used to visualize the results. Tell-F (GGTTTTTGAGGGTGAGGGTGAGGGTGAGGGTGAGGGT) (SEQ ID NO: 1) and ITS18-RC (ATGGGTAAAGGTCAGGGTCACG) (SEQ ID NO: 2) or ITS19-RC (GGTGACGCCCTGTATGCAAA) (SEQ ID NO: 3) were used to detect ligated molecules containing telomere and ITS sequence in the PCR reaction.

The electrophoresis result shows a smear in ligated 3C DNA, but not in non-ligated 3C DNA. This supports the hypothesis since the Tell-F primer can bind to any loci of telomere resulting vary sizes of PCR product.

Furthermore, pairs of oligos were designed to amplify the ITSs themselves as positive controls: ITS19-F (TTTTGGGTATCATGTGTGCATTAGG) (SEQ ID NO: 4) and ITS19-R (AGCCCCGTCTTGCAGTCTTT) (SEQ ID NO: 5); ITS18-F (GCTGATCAGGACGCTTTTGC) (SEQ ID NO: 6) and ITS18-R (TCCTAACGAGGTTCTCCCCA) (SEQ ID NO: 7).

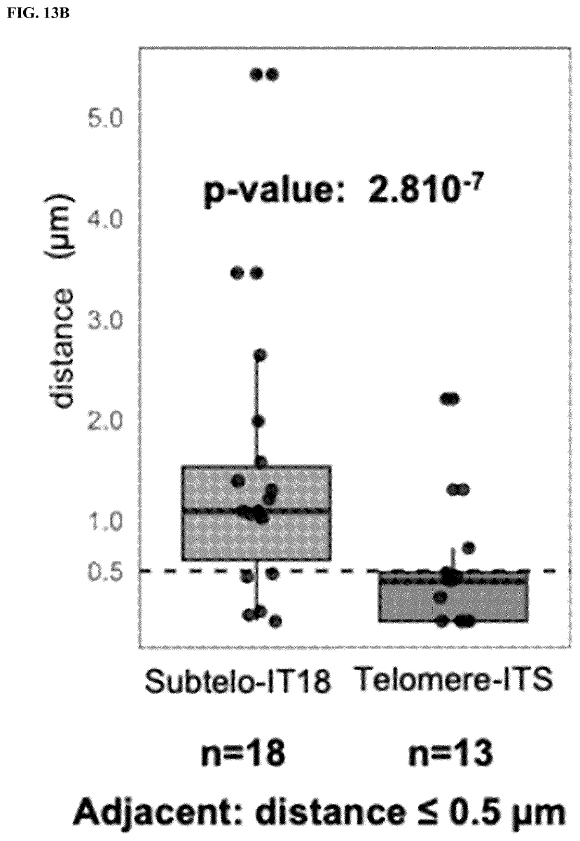

Detection of Telomere-ITS interaction by fluorescence in situ hybridization (FISH) is shown in FIGS. 13A and 13B. Fluorescent in situ hybridization was performed to validate the interaction between telomeres (or subtelomeres) and ITS on chromosome 18. Cells in the interphase were fixed and hybridized with a PNA probe covering telomere regions and a DNA oligo probe covering a selected ITS region (or subtelomere, p-arm) on chromosome 18. The nucleus was imaged, the distance between two foci was measured, and it was determined whether they were adjacent (≤0.5 μm).

It was found that over 50% of the foci between the telomere and ITS are adjacent, while more than 75% of the foci between subtelomere and ITS are separated. The data suggests an interaction between telomere and ITS in a trans manner.

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

The publications discussed herein are provided solely for their disclosure prior to the filing date of the present application. Nothing herein is to be construed as an admission that the present invention is not entitled to antedate such publication by virtue of prior invention.
1. Yanfen Zhu, et. al., Oncogenic extrachromosomal DNA functions as mobile enhancers to globally amplify chromosomal transcription. Cancer Cell. 2021 May 10; 39(5):694-707.e7. doi: 10.1016/j.ccell.2021.03.006. Epub 2021 Apr. 8. PMID: 33836152; PMCID: PMC8119378.
2. Déjardin J, Kingston R E, Purification of proteins associated with specific genomic Loci. Cell. 2009 Jan. 9; 136(1):175-86. doi: 10.1016/j.cell.2008.11.045. PMID: 19135898; PMCID: PMC3395431.
3. Lafontaine D L, Yang L, Dekker J, Gibcus J H. Hi-C 3.0: Improved Protocol for Genome-Wide Chromosome Conformation Capture. Curr Protoc. 2021 July; 1(7):e198. doi: 10.1002/cpz1.198. PMID: 34286910; PMCID: PMC8362010.

What is claimed is:

1. A method of enriching a DNA locus in chromatin, comprising:
preparing chromatin by dual crosslinking using a nucleic acid crosslinking agent and a protein crosslinking agent to produce dual crosslinked chromatin;
shearing the dual crosslinked chromatin using enzymatic digestion or mechanical force;
capturing peptide nucleic acid (PNA) probes and a set of DNA sequences from the chromatin by:
(a) hybridizing PNA probes with attached biotinylated beads to DNA sequences in the dual crosslinked chromatin,
(b) reversing the dual crosslink in the chromatin using a protein digestion enzyme to release the DNA sequences to produce released DNA sequences, and
(c) separating the released DNA sequences by applying a magnet to the biotinylated beads;
extracting from the mixture a fluid containing the released DNA sequences, wherein the fluid does not contain the PNA probes or biotinylated beads;
purifying the released DNA sequences;
generating a ligation-free library with the released DNA sequences, wherein the released DNA sequences include the DNA locus;
indexing the ligation-free library; and
sequencing the ligation-free library using next generation sequencing.

2. The method of claim 1, wherein the protein crosslinking agent is selected from the group consisting of bis(sulfosuccinimidyl) suberate (BS3), disuccinimidyl suberate (DSS), dimethylsuberimidate (DMS), formalin, dimethyladipimidate (DMA), dithio-bis(-succinimidyl propionate) (DSP), disuccinimidyl tartrate (DST), N-hydroxysuccinimide, N-hydroxysulfosuccinimide, ethylene glycol bis(succinimidyl succinate) (EGS), sulfo-ethylene glycol bis(succinimidylsuccinate) (sulfo-EGS), ethylene glycol bis(sulfosuccinimidyl succinate) (SEGS), glutaraldehyde, polyfunctional aziridine, bifunctional carbodiimide, dicyclohexyl carbodiimide, and bis-imidazole-carboxylate compounds.

3. The method of claim 1 or 2, wherein the nucleic crosslinking agent is selected from the group consisting of glutaraldehyde, formaldehyde, psoralen, aminomethyltrioxsalen, cisplatin, disuccinimidyl glutarate, formalin, UV light, mitomycin C, nitrogen mustard, melphalan, 1,3-butadiene diepoxide, cis diaminedichloroplatinum (II), and cyclophosphamide.

4. The method of any one of claims 1 to 3, wherein the protein crosslinking agent is EGS.

5. The method of any one of claims 1 to 4, wherein the nucleic crosslinking agent is methanol-free formaldehyde.

6. The method of any one of claims 1 to 5, wherein the dual crosslinked chromatin is sheared with mechanical force using sonication, nebulization, hydrodynamic shearing, syringe pumping.

7. The method of any one of claims 1 to 6, wherein the protein digestion enzyme is a serine protease, cysteine protease, aspartic protease, threonine protease, metalloprotease, glutamic protease, thiol protease, or a combination thereof.

8. The method of claim 7, wherein the protein digestion enzyme is a serine protease.

9. The method of any one of claims 1 to 8, wherein the released DNA sequences are associated with a transposable element or interspersed repeat.

10. The method of claim 9, wherein the transposable element or interspersed repeat is selected from the list presented in FIG. 14.

11. The method of claim 9, wherein the transposable element or interspersed repeat is a telomere-associated DNA sequence.

12. The method of claim 11, wherein the telomere-associated DNA sequence is a Telomere-Associated Repeat Element (TARE), Subtelomeric Repeat, Interstitial Telomeric Sequence (ITS), Short Interspersed Nuclear Element (SINE), Long Interspersed Nuclear Element (LINE), or satellite DNA.

13. The method of claim 11, wherein the telomere-associated DNA sequence is (CCCTAA)n or (TTAGGG)n.

14. The method of any one of claims 1 to 8, wherein the released DNA sequences are associated with a Retrotransposable Element (RE).

15. The method of claim 14, wherein the RE is an Alu element (ALU).

16. The method of claim 15, wherein the ALU belongs to a subfamily selected from the group consisting of AluJ, AluS, AluY, AluSc, AluSq, AluSp, AluYb8, AluYa5, AluYh9, and AluJo.

17. The method of any one of claims 1 to 16, wherein the PNA probe comprises a complementary nucleotide sequence to a transposable element, interspersed repeat, or retrotransposable element.

18. A method of capturing telomere-associated DNA loci from chromatin, comprising:
crosslinking the chromatin by mixing chromatin with a nucleic acid crosslinking agent and a protein crosslinking agent to produce dual crosslinked chromatin;
shearing the dual crosslinked chromatin using enzymatic digestion or mechanical force;
capturing PNA probes and a set of DNA sequences from the chromatin by:
(a) hybridizing PNA probes with biotinylated beads to DNA sequences in the dual crosslinked chromatin,
(b) reversing the dual crosslink in the chromatin using a protein digestion enzyme to release the DNA sequences to produce released DNA sequences, and
(c) separating the released DNA sequences by applying a magnet to the biotinylated beads; and
extracting from the mixture a fluid containing the released DNA sequences, wherein the fluid does not contain the PNA probes or biotinylated beads, and wherein the released DNA sequences include the telomere-associated DNA loci.

19. The method of claim 18, wherein the protein crosslinking agent is selected from the group consisting of bis(sulfosuccinimidyl) suberate (BS3), disuccinimidyl suberate (DSS), dimethylsuberimidate (DMS), formalin, dimethyladipimidate (DMA), dithio-bis(-succinimidyl propionate) (DSP), disuccinimidyl tartrate (DST), N-hydroxysuccinimide, N-hydroxysulfosuccinimide, ethylene glycol bis(succinimidyl succinate) (EGS), sulfo-ethylene glycol bis(succinimidylsuccinate) (sulfo-EGS), ethylene glycol bis(sulfosuccinimidyl succinate) (SEGS), glutaraldehyde, polyfunctional aziridine, bifunctional carbodiimide, dicyclohexyl carbodiimide, and bis-imidazole-carboxylate compounds.

20. The method of claim 18 or 19, wherein the nucleic crosslinking agent is selected from the group consisting of glutaraldehyde, formaldehyde, psoralen, aminomethyltrioxsalen, cisplatin, disuccinimidyl glutarate, formalin, UV light, mitomycin C, nitrogen mustard, melphalan, 1,3-butadiene diepoxide, cis diaminedichloroplatinum (II), and cyclophosphamide.

21. The method of any one of claims 18 to 20, wherein the protein crosslinking agent is EGS.

22. The method of any one of claims 18 to 21, wherein the nucleic crosslinking agent is methanol-free formaldehyde.

23. The method of any one of claims 18 to 22, wherein the dual crosslinked chromatin is sheared with mechanical force using sonication, nebulization, hydrodynamic shearing, syringe pumping.

24. The method of any one of claims 18 to 23, wherein the protein digestion enzyme is a serine protease, cysteine protease, aspartic protease, threonine protease, metalloprotease, glutamic protease, thiol protease, or a combination thereof.

25. The method of claim 24, wherein the protein digestion enzyme is a serine protease.


SEQUENCE LISTING (7 sequences)

SEQ ID NO: 1 - DNA, 37 bp, synthetic construct
  source 1..37: mol_type = other DNA
ggtttttgagggtgagggtgagggtgagggtgagggt

SEQ ID NO: 2 - DNA, 22 bp, synthetic construct
  source 1..22: mol_type = other DNA
atgggtaaaggtcagggtcacg

SEQ ID NO: 3 - DNA, 20 bp, synthetic construct
  source 1..20: mol_type = other DNA
ggtgacgccctgtatgcaaa

SEQ ID NO: 4 - DNA, 25 bp, synthetic construct
  source 1..25: mol_type = other DNA
ttttgggtatcatgtgtgcattagg

SEQ ID NO: 5 - DNA, 20 bp, synthetic construct
  source 1..20: mol_type = other DNA
agccccgtcttgcagtcttt

SEQ ID NO: 6 - DNA, 20 bp, synthetic construct
  source 1..20: mol_type = other DNA
gctgatcaggacgcttttgc

SEQ ID NO: 7 - DNA, 20 bp, synthetic construct
  source 1..20: mol_type = other DNA
tcctaacgaggttctcccca